(19) United States
(12) Patent Application Publication
(10) Pub. No.: US 2026/0364582 A1
(43) Pub. Date: Nov. 19, 2026

(54) CATALYST, MODIFIED COMOPOUND, AND METHOD OF PREPARING MODIFIED COMPOUND

(71) Applicant: OKINAWA INSTITUTE OF SCIENCE AND TECHNOLOGY SCHOOL CORPORATION, Kunigami-gun Okinawa (JP)

(72) Inventors: Eugene KHASKIN, Kunigami-gun, Okinawa (JP); Shubham DEOLKA, Kunigami-gun, Okinawa (JP)

(73) Assignee: OKINAWA INSTITUTE OF SCIENCE AND TECHNOLOGY SCHOOL CORPORATION, Kunigami-gun Okinawa (JP)

(21) Appl. No.: 19/488,692

(22) PCT Filed: Jul. 5, 2024
(86) PCT No.: PCT/JP2024/024514

§ 371 Date: Nov. 28, 2025

(30) Foreign Application Priority Data
Jul. 7, 2023 (JP) 2023-112580

(51) Int. Cl. B01J 31/12; C07C 41/30; C07D 207/33; C07D 207/333; C07D 209/10; C07D 209/12; C07D 209/14; C07D 209/18; C07D 233/60; C07D 239/60; C07D 241/12; C07D 307/38; C07D 403/06; C07K 1/113; C07K 5/065; C07K 5/083; C07K 5/103; C07K 5/107; C07K 7/06; C07K 7/08

(52) U.S. Cl. CPC B01J 31/122; C07C 41/30; C07D 207/33; C07D 207/333; C07D 209/10; C07D 209/12; C07D 209/14; C07D 209/18; C07D 233/60; C07D 239/60; C07D 241/12; C07D 307/38; C07D 403/06; C07K 1/113; C07K 5/06078; C07K 5/0808; C07K 5/1008; C07K 5/1016; C07K 7/06; C07K 7/08; B01J 2231/4205; B01J 2531/847

(57) ABSTRACT

Provided is a catalyst that can be used in a variety of reactions involving a formation of a carbon-carbon bond. The catalyst is represented by a formula (I). In formula (I), R¹ is selected from the group consisting of a hydrogen atom, a fluorine atom, a hydrocarbon group and a heterocyclic group; R² is selected from the group consisting of a hydrogen atom, a fluorine atom, a hydrocarbon group and a heterocyclic group; each R³ is independently selected from the group consisting of CH₂, CHF and CF₂; R⁴ and R⁵ are the same or different and each is a polar organic solvent comprising at least one selected from a nitrogen atom, an oxygen atom, a sulfur atom and a halogen atom; n is an integer larger than 0; and dashed lines connecting Ni (II) and R⁴ and R⁵ are coordinate bonds.

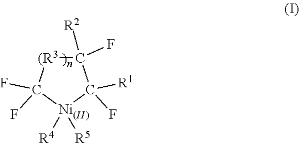

### TECHNICAL FIELD

The present disclosure relates to a catalyst used for a reaction involving a formation of a carbon-carbon bond, a modified compound prepared by a reaction involving a formation of a carbon-carbon bond, and a method of preparing a modified compound.

### BACKGROUND

The modification of existing drugs and bioactive molecules has been established as a strategy to alter their activity, improve stability to metabolic degradation, and assist in their passage through the blood/brain barrier. These effects can vary greatly depending on the type of the modification.

Therefore, developing a simple and general catalytic protocol that can be used for a wide variety of modifications is an important goal.

Over the past decade, nickel has emerged as a cheaper and more sustainable alternative to second and third-row metals for a wide variety of chemical transformations.

For example, NPLs 1 and 2 disclose ligand-free, Ni-catalyzed perfluoroalkylation protocols.

### CITATION LIST

### Non Patent Literature

NPL 1: Deolka, S.; Govindarajan, R.; Vasylevskyi, S.; Roy, M. C.; Khusnutdinova, J. R.; Khaskin, E., Ligand-free nickel catalyzed perfluoroalkylation of arenes and heteroarenes. Chem. Sci. 2022, 13 (44), 12971-12979.
NPL 2: Shreiber, S. T.; Vicic, D. A., Solvated Nickel Complexes as Stoichiometric and Catalytic Perfluoroalkylation Agents. Angew. Chem. Int. Ed. 2021, 60 (33), 18162-18167.

### SUMMARY

### Technical Problem

However, the conventional ligand-free, Ni-catalyzed perfluoroalkylation protocols described above have a significant downside in that a different pre-catalyst (CH₃CN)₂Ni—(CₙF₂ₙ₊₁)₂ has to be prepared for each perfluoroalkyl —CₙF₂ₙ₊₁ group.

Accordingly, one object of the present disclosure is to provide a catalyst that can be used in a variety of reactions involving a formation of a carbon-carbon bond.

Another object of the present disclosure is to provide a modified compound that can be prepared by a reaction involving a formation of a carbon-carbon bond.

Another object of the present disclosure is to provide a method of preparing a modified compound that can be prepared by a reaction involving a formation of a carbon-carbon bond.

### Solution to Problem

The inventors conducted diligent investigation with the aim of solving the problem set forth above. The inventor discovered that the specific cyclometalated nickel complex can catalyze a reaction involving a carbon-carbon bond formation, and, in this manner, completed the present disclosure.

The gist of the present disclosure is as follows:
[1]A catalyst used in a reaction involving a formation of a carbon-carbon bond, wherein the catalyst is represented by a formula (I):

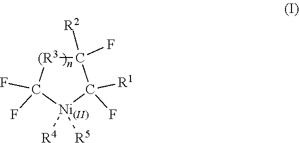

wherein
R¹ is selected from the group consisting of a hydrogen atom, a fluorine atom, a hydrocarbon group and a heterocyclic group;
R² is selected from the group consisting of a hydrogen atom, a fluorine atom, a hydrocarbon group and a heterocyclic group;
each R³ is independently selected from the group consisting of CH₂, CHF and CF₂;
R⁴ and R⁵ are the same or different and each is a polar organic solvent comprising at least one selected from a nitrogen atom, an oxygen atom, a sulfur atom and a halogen atom;
n is an integer larger than 0; and
dashed lines connecting Ni (II) and R⁴ and R⁵ are coordinate bonds.
[2] The catalyst according to [1], wherein R¹ is a fluorine atom.
[3] The catalyst according to [1] or [2], wherein R² is a fluorine atom.
[4] The catalyst according to any one of [1] to [3], wherein each R³ is CF₂.
[5] The catalyst according to any one of [1] to [4], wherein R⁴ and R⁵ are independently selected from the group consisting of an alcohol, an amide, a ketone, an ether, a nitrile, an alkyl halide and a sulfoxide.
[6] The catalyst according to any one of [1] to [5], wherein both R⁴ and R⁵ are CH₃CN.
[7] The catalyst according to any one of [1] to [6], wherein n is an integer from 1 to 3.
[8] The catalyst according to any one of [1] to [7], wherein the carbon-carbon bond is formed between a carbon atom of a substrate and a fluorocarbon group of a fluorocarbon compound.
[9] The catalyst according to [8], wherein the substrate comprises at least one of an aromatic carbon ring and an aromatic heterocyclic ring.
[10] The catalyst according to [8] or [9], wherein the fluorocarbon compound comprises at least one —(CF₂)ₘ—, wherein m is an integer larger than 0.
[11] The catalyst according to any one of [8] to [10], wherein the fluorocarbon compound is represented by formula (II-1), (II-2), (II-3) or (II-4):

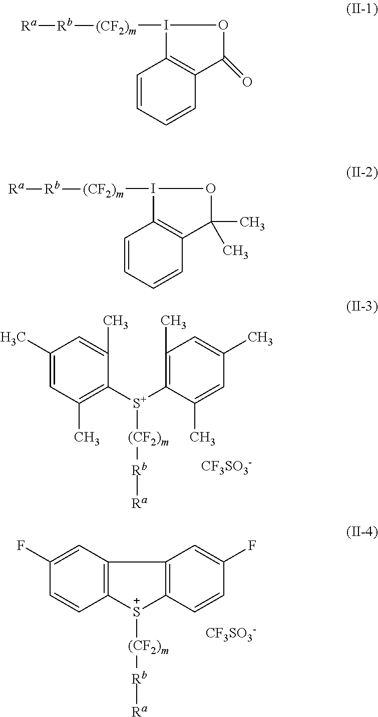

wherein
R^{a} is selected from the group consisting of a fluorine atom, an aryl group and a heteroaryl group, wherein the aryl group and the heteroaryl group are each optionally substituted by one or more substituents selected from the group consisting of a heteroatom and an alkyl group;
R^{b} is a single bond or an oxygen atom; and
m is an integer larger than 0.
[12]A modified compound prepared by a reaction involving a formation of a carbon-carbon bond, wherein the reaction uses a catalyst represented by a formula (I):

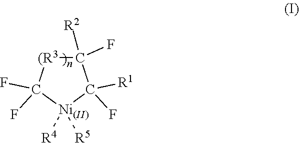

wherein
R¹ is selected from the group consisting of a hydrogen atom, a fluorine atom, a hydrocarbon group and a heterocyclic group;
R² is selected from the group consisting of a hydrogen atom, a fluorine atom, a hydrocarbon group and a heterocyclic group;
each R³ is independently selected from the group consisting of CH₂, CHF and CF₂;
R⁴ and R⁵ are the same or different and each is a polar organic solvent comprising at least one selected from a nitrogen atom, an oxygen atom, a sulfur atom and a halogen atom;
n is an integer larger than 0; and
dashed lines connecting Ni (II) and R⁴ and R⁵ are coordinate bonds.
[13]A method of preparing a modified compound represented by formula (IV), comprising:
reacting a substrate represented by formula (III) with a fluorocarbon compound represented by formula (II-1), (II-2), (II-3) or (II-4) as defined above, in the presence of the catalyst as defined above to obtain the modified compound represented by formula (IV):

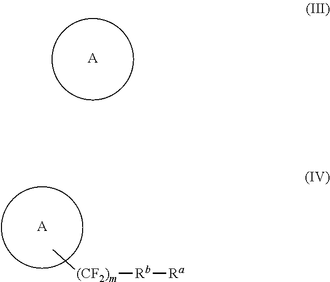

wherein
A is selected from the group consisting of an aromatic carbon ring and an aromatic heterocyclic ring, wherein the aromatic carbon ring and the aromatic heterocyclic ring may be substituted by one or more substituents;
A may be a part of a peptide or protein; and
R^{a}, R^{b} and m are as defined above.
[14] The method according to [13], wherein A is selected from the group consisting of a benzene ring and an aromatic heterocyclic ring having 5 to 12 ring atoms.
[15] The method according to [13] or [14], wherein A is an indole ring of a tryptophan residue in a peptide or a protein.

### Advantageous Effects

According to the present disclosure, it is possible to provide a catalyst that can be used in a variety of reactions involving a formation of a carbon-carbon bond.

Moreover, according to the present disclosure, it is possible to provide a modified compound that can be prepared by a reaction involving a formation of a carbon-carbon bond.

Furthermore, according to the present disclosure, it is possible to provide a method of preparing a modified compound that can be prepared by a reaction involving a formation of a carbon-carbon bond.

### BRIEF DESCRIPTION OF DRAWINGS

In the accompanying drawings:

FIG. 1 shows an example of a modified compound prepared by a reaction using the presently disclosed catalyst;

FIG. 2 shows another example of modified compounds prepared by a reaction using the presently disclosed catalyst;

FIG. 3 shows another example of a modified compounds prepared by a reaction using the presently disclosed catalyst.

### DETAILED DESCRIPTION

The presently disclosed techniques will be described in detail below.

### TERMS

“Alkyl group” may be linear or branched, and the number of carbon atoms may generally be 1 to 20. “Alkyl group” includes methyl, ethyl, n-propyl, isopropyl, n-butyl, sec-butyl, tert-butyl, n-pentyl, sec-pentyl, tert-pentyl, neopentyl, amyl, hexyl, heptyl, octyl, nonyl, decyl, undecyl, dodecyl, etc.

“Alkenyl group” may be linear or branched, and the number of carbon atoms may generally be 2 to 20. “Alkenyl group” includes vinyl, allyl, butenyl, butadienyl, pentel, hexenyl, heptenyl, octenyl, nonenyl, decenyl, undecenyl, dodecenyl, etc.

“Alkynyl group” may be linear or branched, and the number of carbon atoms may generally be 2 to 20. “Alkynyl group” includes ethynyl, propagyl, butynyl, pentynyl, hexynyl, heptynyl, octynyl, nonynyl, decynyl, undecynyl, dodecynyl, etc.

“Cycloalkyl group” may be a monocyclic group or a polycyclic group, and the number of carbon atoms may generally be 3 to 30. “Cycloalkyl group” includes cyclopentyl, cyclohexyl, cycloheptyl, adamantyl, methylcyclohexyl, ethylcyclohexyl, etc.

“Alkylene group” may be linear or branched, and the number of carbon atoms may generally be 1 to 20. “Alkylene group” includes methylene, ethylene, propylene, butylene, pentylene, hexylene, heptylene, octylene, nonylene, decylene, undecylene, dodecylene, etc.

“Alkenylene group” may be linear or branched, and the number of carbon atoms may generally be 2 to 20 carbon atoms. “Alkenylene group” includes vinylene, propenylene, butenylene, butadienylene, octenylene, decenylene, dodecenylene, etc.

“Alkynylene group” may be linear or branched, and the number of carbon atoms may generally be 2 to 20 carbon atoms. “Alkynylene group” includes ethynylene, propynylene, butynylene, pentynylene, hexynylene, heptynylene, octynylene, noninylene, decynylene, undecinylene, dodecinylene group, etc.

“Alkoxy group” is an alkyl-O—, and the description of the alkyl group above applies to the alkyl moiety. “Alkoxy group” includes methoxy, ethoxy, propoxy, isopropoxy, n-butoxy, sec-butoxy, tert-butoxy, allyloxy, etc.

“Aryl group” is a monovalent aromatic hydrocarbon group, and the number of carbon atoms may generally be 6 to 20 carbon atoms. “Aryl group” includes phenyl, naphthyl, biphenyl, phenanthryl, etc.

“Heteroaryl group” is a monovalent aromatic heterocyclic group, and the number of ring atoms may generally be 4 to 10. Hetero atoms may be nitrogen, oxygen and sulfur.

“Heteroaryl group” includes pyridinyl, furanyl, thiophenyl, etc.

“Arylene group” is a divalent group corresponding to an aryl group.

“Heteroarylene group” is a divalent group corresponding to a heteroaryl group.

“Heteroatom” is any atom other than hydrogen and carbon atoms.

“Heteroatom group” is a monovalent group containing at least one heteroatom.

“Heteroatom group” includes carboxy group, hydroxy group, sulfo group, phosphate group, amino group, etc.

“Halogen atom” involves fluorine, chlorine, bromine and iodine atoms.

“Carbon-carbon bond” is a covalent bond between two carbon atoms. The “carbon-carbon bond” can be a single bond, a double bond, or a triple bond, but preferably a single bond.

“Fluorocarbon group” is a monovalent or divalent group containing at least one bond connecting a carbon atom and a fluorine atom. “Fluorocarbon group” includes —CF₂—, —CFH—, —CF₃, —CF₂H, —CFH₂, —etc.

“Aromatic carbon ring” may be monocyclic or polycyclic, and the number of ring atoms may generally be 6 to 14. “Aromatic carbon ring” includes benzene, naphthalene, anthracene, etc. “Aromatic carbon ring” may be substituted by one or more substituents. The substituent includes, for example, an alkyl group, an alkoxy group, an aryl group, an aralkyl group and a heteroatom group, which are each optionally substituted by one or more substituents such as an amino group, a halogen atom, a carboxyl group, a hydroxy group, etc. “Aromatic carbon ring” may be a part of a peptide or protein.

“Aromatic heterocyclic ring” contains at least one atom selected from nitrogen, oxygen and sulfur as a ring atom. “Aromatic heterocyclic ring” may be monocyclic or polycyclic, and the number of ring atoms may generally be 5 to 14. Monocyclic ring includes pyrrole, furan, thiophene, pyrazole, imidazole, oxazole, thiazole, isoxazole, pyridine, pyrimidine, pyrazine, pyridazine, triazine, etc. Polycyclic ring includes indole ring, isoindole, benzofuran, benzoxazole, indazole, benzimidazole, purine, etc.

“Aromatic heterocyclic ring” may be substituted by one or more substituents. The substituent includes, for example, an alkyl group, an alkoxy group, an aryl group, an aralkyl group and a heteroatom group, which are each optionally substituted by one or more substituents such as an amino group, a halogen atom, a carboxyl group, a hydroxy group, etc. “Aromatic heterocyclic ring” may be a part of a peptide or protein.

### (Catalyst)

The presently disclosed catalyst is represented by formula (I):

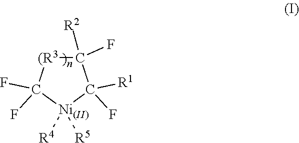

wherein, R¹ is selected from the group consisting of a hydrogen atom, a fluorine atom, a hydrocarbon group and a heterocyclic group;
R² is selected from the group consisting of a hydrogen atom, a fluorine atom, a hydrocarbon group and a heterocyclic group;
each R³ is independently selected from the group consisting of CH₂, CHF and CF₂;
R⁴ and R⁵ are the same or different and each is a polar organic solvent comprising at least one selected from a nitrogen atom, an oxygen atom, a sulfur atom and a halogen atom;
n is an integer larger than 0; and
dashed lines connecting Ni (II) and R⁴ and R⁵ are coordinate bonds.
   Note that in a case in which n in formula (I) is larger than 1, each R³ may be the same or different.

In formula (I), R¹ is selected from the group consisting of a hydrogen atom (H), a fluorine atom (F), a hydrocarbon group and a heterocyclic group.

Examples of hydrocarbon groups constituting R¹ include, but are not specifically limited to, an alkyl group, an alkenyl group, an alkynyl group, a cycloalkyl group, and an aryl group.

Examples of heterocyclic groups constituting R¹ include, but are not specifically limited to, a thienyl group, a pyrrolyl group, a furyl group, a pyridyl group, a piperidyl group, a quinolyl group, an isoquinolyl group, a triazyl group, a quinazolinyl group, an imidazolyl group, etc.

Preferably, R¹ in formula (I) is a fluorine atom.

In formula (I), R² is selected from the group consisting of a hydrogen atom (H), a fluorine atom (F), a hydrocarbon group and a heterocyclic group.

The hydrocarbon groups that can constitute R² include the same hydrocarbon groups that can constitute R¹.

The heterocyclic groups that can constitute R² include the same heterocyclic groups that can constitute R¹.

Preferably, R² in formula (I) is a fluorine atom.

In formula (I), each R³ is independently selected from the group consisting of CH₂, CHF and CF₂. Preferably, each R³ in formula (I) is CF₂.

In formula (I), R⁴ and R⁵ are the same or different and each is a polar organic solvent comprising at least one selected from a nitrogen atom (N), an oxygen atom (O), a sulfur atom (S) and a halogen atom. Examples of such polar organic solvents include, but are not specifically limited to, alcohols such as methanol (CH₃OH), ethanol (CH₃CH₂OH), 1-propanol (CH₃CH₂CH₂OH), 2-propanol (CH₃CH(OH)CH₃) and 1-butanol (CH₃CH₂CH₂CH₂OH); ketones such as acetone (CH₃C(═O)CH₃); sulfoxides such as dimethyl sulfoxide (CH₃S(═O)CH₃); nitriles such as acetonitrile (CH₃CN); ethers such as tetrahydrofuran (C₄H₈O) and diethyl ether (CH₃CH₂OCH₂CH₃); alkyl halides such as dichloromethane (CH₂Cl₂); and amides such as N,N-dimethylformamide (HC(═O)N(CH₃)₂). Of these, acetonitrile (CH₃CN) is preferable.

Note that R⁴ and R⁵ in formula (I) may be the same or different, but are preferably the same.

In formula (I), n is an integer larger than 0, preferably an integer from 1 to 3, more preferably from 1 to 2, and most preferably 1.

Dashed lines connecting Ni (II) and R⁴ and R⁵ in formula (I) represent coordinate bonds in which an electron pair is supplied from an electron pair donor (i.e. a nitrogen atom (N), an oxygen atom (O), a sulfur atom (S) or a halogen atom in R⁴ and R⁵) to an electron pair acceptor (i.e. Ni (II)).

### <Reaction Involving a Formation of a Carbon-Carbon Bond>

The presently disclosed catalyst set forth above can be used in a reaction involving a formation of a carbon-carbon bond. The reaction, in which the presently disclosed catalyst can be used, is not specifically limited, so long as it involves a formation of a carbon-carbon bond.

In some embodiments of the present disclosure, the carbon-carbon bond is formed between a carbon atom of a substrate and a fluorocarbon group of a fluorocarbon compound.

### <<Substrate>>

The substrate is not specifically limited, so long as it has at least one carbon atom that can form a carbon-carbon bond with a fluorocarbon group of a fluorocarbon compound through a reaction using the presently disclosed catalyst.

The substrate preferably comprises at least one of an aromatic carbon ring and an aromatic heterocyclic ring. Among aromatic carbon rings, benzene ring is preferable. Among aromatic heterocyclic rings, an aromatic heterocyclic ring having 5 to 12 ring atoms is preferable, an aromatic heterocyclic ring having 5 to 9 ring atoms is more preferable, and a furan ring, a pyrrole ring, a pyrazine ring, a pyrimidine ring and an indole ring are even more preferable.

In some embodiments of the present disclosure, the substrate is represented by formula (V):

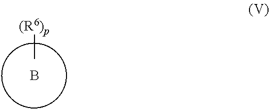

In formula (V), B is an unsubstituted aromatic carbon ring or aromatic heterocyclic ring.

R⁶ in formula (V) is selected from the group consisting of an alkyl group, an alkoxy group, an aryl group, an aralkyl group and a heteroatom group, wherein the alkyl group, the alkoxy group, the aryl group, the aralkyl group and the heteroatom group are each optionally substituted by one or more substituents selected from the group consisting of an amino group, a halogen atom, a carboxyl group, and a hydroxy group. In a case in which there are more than 2 substituents, they may be the same or different.

The “p” in formula (V) is an integer of not less than 0, and preferably not less than 1, and preferably less than 6, more preferably less than 5, even more preferably less than 4, and most preferably less than 3. In a case in which p in formula (V) is not less than 2, each R⁶ may be the same or different.

In some embodiments of the present disclosure, the substrate is a peptide or protein comprising at least one selected from a tryptophan residue, a histidine residue, a tyrosine residue and a phenylalanine residue. Preferably, the substrate is a peptide or protein comprising at least one tryptophan residue. The number of amino acid residues in the peptide or protein is not specifically limited. The number of amino acid residues in the peptide or protein is preferably 200 or less, more preferably 100 or less, and even more preferably 50 or less. Preferably, the protein or peptide has a tryptophan residue, a histidine residue, a tyrosine residue or a phenylalanine residue at the N-terminus or C-terminus of a peptide or protein.

### <<Fluorocarbon Compound>>

The fluorocarbon compound is a compound comprising at least one fluorocarbon group. The fluorocarbon group in the fluorocarbon compound can form a carbon-carbon bond with a carbon atom of a substrate through a reaction using the presently disclosed catalyst.

The fluorocarbon compound preferably comprises at least one —(CF₂)ₘ—, wherein m is an integer larger than 0. Although no specific limitations are placed on the upper limit for m, the upper limit can be set, for example, as 50 or less, 40 or less, or 30 or less.

The fluorocarbon compound preferably comprises at least one perfluoroalkyl group. Examples of perfluoroalkyl groups include, but are not specifically limited to, CF₃, C₂F s, C₃F₇, C₄F₉, C₅F₁₁, C₆F₁₃, C₇F₁₅, C₈F₁₇, etc.

The fluorocarbon compound may be a fluoroalkylating agent (e.g., a trifluoroalkylating agent). Examples of fluorocarbon compounds include, but are not specifically limited to, an acid Togni reagent represent by formula (II-1), an alcohol Togni reagent represent by formula (II-2), an Umemoto reagent represent by formula (II-3), and an Umemoto TT reagent represented by formula (II-4):

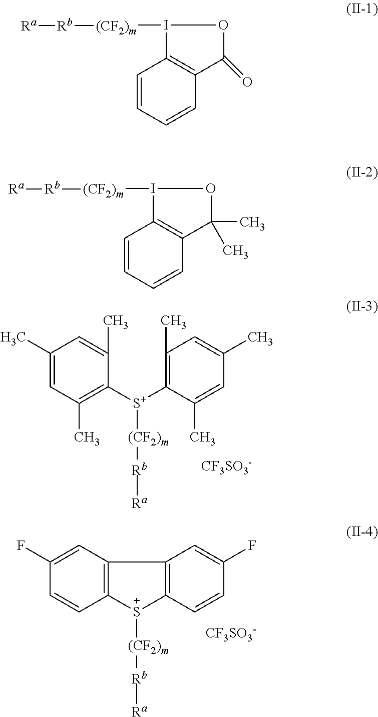

In formula (II-1), (II-2), (II-3) and (II-4), R^{a} is selected from the group consisting of a fluorine atom, an aryl group and a heteroaryl group, wherein the aryl group and the heteroaryl group are each optionally substituted by one or more substituents. The substituent is selected from the group consisting of a heteroatom and an alkyl group, and preferably a halogen atom. In a case in which there are more than 2 substituents, they may be the same or different. Note that the fluorocarbon compound may form a salt.

Preferred examples of R^{a} include, for example, a fluorine atom and those represented by formula (a-1) to (a-3) shown below:

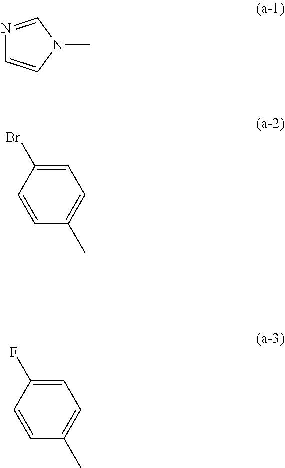

R^{b} in formula (II-1), (II-2), (II-3) and (II-4) is a single bond or an oxygen atom.

The “m” in formula (II-1), (II-2), (II-3) and (II-4) is as defined in —(CF₂)ₘ—.

The fluorocarbon compound is preferably an acid Togni reagent or an alcohol Togni reagent, and more preferably an acid Togni reagent.

### <Preparation Method of Catalyst>

The presently disclosed catalyst can be synthesized by a commonly known method such as those described in the literature below.

Kaplan, P. T.; Xu, L.; Chen, B.; McGarry, K. R.; Yu, S.; Wang, H.; Vicic, D. A., “Mild, Safe, and Versatile Reagents for (CF₂), Transfer and the Construction of Fluoroalkyl-Containing Rings.” Organometallics 2013, 32 (24), 7552-7558.

### (Modified Compound)

The presently disclosed modified compound is prepared by a reaction that involves a formation of a carbon-carbon bond using the presently disclosed catalyst set forth above. The reaction used for the preparation of the modified compound is not specifically limited, so long as it involves a formation of a carbon-carbon bond using the presently disclosed catalyst.

In some embodiments of the present disclosure, the presently disclosed modified compound is prepared by a reaction in which a carbon-carbon bond is formed between a carbon atom of a substrate and a fluorocarbon group of a fluorocarbon compound in the presence of the presently disclosed catalyst.

In the aforementioned embodiment, the substrate and the fluorocarbon compound can be those described above in the “Catalyst” section.

In a case in which the substrate is not a peptide or a protein, the amount of a fluorocarbon compound relative to the amount of a substrate used in the reaction is preferably 0.8 molar equivalent or more, and more preferably 1 molar equivalent or more, and preferably 2 molar equivalent or less, and more preferably 1.5 molar equivalent or less.

In a case in which the substrate is a peptide or a protein, the amount of a fluorocarbon compound relative to the amount of a substrate used in the reaction is preferably 0.8 molar equivalent or more, and more preferably 1 molar equivalent or more, and preferably 5 molar equivalent or less, and more preferably 4 molar equivalent or less.

When the amount of a fluorocarbon compound relative to the amount of a substrate is not less than the lower limit set forth above, the reaction yield can increase. When the amount of a fluorocarbon compound relative to the amount of a substrate is not more than the upper limit set forth above, reagent cost can be reduced.

The amount of a catalyst used in the reaction when the amount of a substrate used in the reaction is taken to be 100 mol % is preferably 5 mol % or more, and more preferably 7.5 mol % or more, and preferably 20 mol % or less, and more preferably 15 mol % or less. When the amount of a catalyst is 5 mol % or more, the reaction yield can increase. On the other hand, when the amount of a catalyst is 20 mol % or less, reagent cost can be reduced.

The reaction is usually performed in the presence of a solvent. The solvent used includes, but is not limited to, the polar organic solvents described above in the “Catalyst” section.

In addition to the catalyst, the substrate, the fluorocarbon compound and the solvent set for above, additives may be added to the reaction mixture. Additives are not specifically limited so long as they do not affect the reaction catalyzed by the presently disclosed catalyst.

The reaction temperature is not specifically limited, but the reaction temperature is preferably from 10 °C to 120 °C, and more preferably 20 °C to 100 °C. The reaction time can be set as appropriate depending on the desired reaction yield, but the reaction time is preferably 12 hours or more, more preferably 24 hours or more, and even more preferably 36 hours or more. From a viewpoint of increasing reaction yield, it is preferable that the reaction is performed under nitrogen atmosphere.

### (Method of Preparing Modified Compound)

The presently disclosed method of preparing a modified compound represented by formula (IV) comprises reacting a substrate represented by formula (III) with a fluorocarbon compound represented by formula (II-1), (II-2). (II-3) or (II-4) set forth above, in the presence of the presently disclosed catalyst set forth above to obtain the modified compound represented by formula (IV).

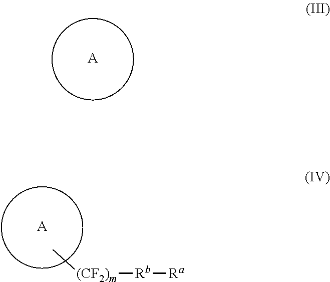

“A” in formula (III) and (IV) is selected from the group consisting of an aromatic carbon ring and an aromatic heterocyclic ring, wherein the aromatic carbon ring and the aromatic heterocyclic ring may be substituted by one or more substituents. Examples of substituents include, but are not specifically limited to, an alkyl group, an alkoxy group, an aryl group, an aralkyl group and a heteroatom group, which are each optionally substituted by one or more substituents such as an amino group, a halogen atom, a carboxyl group, a hydroxy group, etc.

Among aromatic carbon rings that can constitute “A”, a benzene ring is preferable. Among aromatic heterocyclic rings that can constitute “A”, an aromatic heterocyclic ring having 5 to 12 ring atoms is preferable, an aromatic heterocyclic ring having 5 to 9 ring atoms is more preferable, and a furan ring, pyrrole ring, a pyrazine ring, a pyrimidine ring and an indole ring are even more preferable.

“A” in formula (III) and (IV) may be a part of a peptide or protein. Specifically, “A” can be a part of a peptide or protein comprising at least one selected from a tryptophan residue, a histidine residue, a tyrosine residue and a phenylalanine residue. Preferably, “A” is an indole ring of a tryptophan residue in a peptide or protein, and the reaction attaches R^{a}—R^{b}—(CF₂)ₘ— to 2-position of an indole ring in a tryptophan residue shown below.

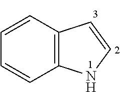

In some embodiments of the present disclosure, the method of preparing a modified compound comprises reacting a substrate represented by formula (V) with a fluorocarbon compound represented by formula (II-1), (II-2), (II-3) or (II-4) set forth above, in the presence of the presently disclosed catalyst set forth above to obtain the modified compound represented by formula (VI).

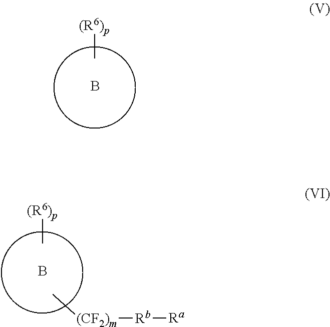

“B”, R⁶, R^{a}, R^{b}, “p” and “m” in formula (V) and (VI) are as defined above.

The presently disclosed method of preparing a modified compound represented by formula (IV) can further comprise purifying the resultant modified compound. The purification method used to purify the modified compound may be, but is not specifically limited to, a known purification method such as solvent extraction or column chromatography.

Also note that purification of the modified compound may be repeated multiple times.

The present disclosure also relates to a use of the catalyst represented by formula (I) set forth above in a reaction involving a formation of a carbon-carbon bond.

### EXAMPLES

The following provides a more specific description of the present disclosure based on examples.

The materials and analytical methods used in the experiments are as follows.

### Example 1

### Materials

All chemicals unless noted otherwise were purchased from commercial suppliers (TCI, Sigma-Aldrich, CF-Plus chemicals and Nacalai Tesque) and used without purification. Anhydrous solvents were dispensed from an MBRAUN solvent purification system and degassed prior to use. Anhydrous deuterated solvents were purchased from Eurisotop/Aldrich/Merck and stored over 4 angstroms molecular sieves. 1-trifluoromethyl-1,2-benziodoxol-3(1H)-one (contains 60% Diatomaceous earth). Metal precursor (C₄F₈)Ni(MeCN)₂ was prepared according to Kaplan, P. T. et al., “Mild, Safe, and Versatile Reagents for (CF₂), Transfer and the Construction of Fluoroalkyl-Containing Rings.” Organometallics 2013, Vol. 32 (24), p. 7552-7558.

Synthesis of peptides were performed under the analogue condition reported by Saze and coworkers in Chapter 6, Plant Epigenetics and Epigenomics.

### <Sample Characterization>

NMR spectra were measured on JEOL ECZ600R 600 MHz, JEOL ECZ400S 400 MHz and Bruker Avance III Neo 500 MHz (CryoProbe) spectrometers. The following abbreviations are used for describing NMR spectra: s (singlet), d (doublet), t (triplet), br. s (broad singlet), vd (virtual doublet), vt (virtual triplet), tq (triplet of quartets), qt (quartet of triplets), and br (broad).

To reference the ¹⁹F NMR spectra, α,α,α-trifluorotoluene was used and the corresponding chemical shift was appeared at −61.04 ppm in DMSO solvent at ambient conditions.

Electrospray Ionization High-Resolution Mass Spectrometry (ESI-HRMS) measurements were performed on a Thermo Scientific ETD apparatus.

Absorbance UV/vis spectra were collected using an Agilent Cary 60 instrument.

All reactions were performed under a nitrogen atmosphere unless indicated otherwise. NMR yield was based on integration against α,α,α-trifluorotoluene as an internal standard.

### Example 2 (Development of Catalytic Protocol)

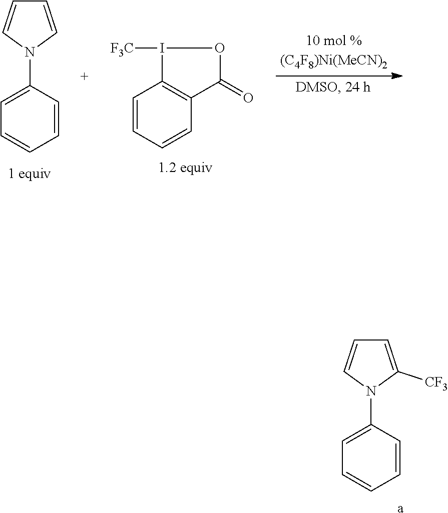

Inside the glovebox, a 20 mL vial equipped with a magnetic stirring bar was charged with 1-phenylpyrrole (10.0 mg, 0.069 mmol), 1-trifluoromethyl-1,2-benziodoxol-3(1H)-one (66.2 mg, 0.082 mmol, acid Togni reagent) and (C₄F₈)Ni(MeCN)₂ (2.2 mg, 6.9 mol). Then anhydrous DMSO (dimethyl sulfoxide) or other solvent (1 mL) was added. The reaction mixture was stirred vigorously at room temperature for 24 hours. After reaction completion, α,α,α-trifluorotoluene (8.5 L, 0.069 mmol) was added as a standard and the yield was determined by ¹⁹F NMR spectroscopy (Table 1).

The first catalytic experiment was attempted with acid CF₃-Togni reagent), 10 mol % of 1, and 1-phenylpyrrole, under air in DMSO. Already with these conditions we were able to observe 15% of functionalized product (Table 1, entry 1). Performing the same reaction under nitrogen atmosphere gave a more practical 78% yield (entry 2) after 24 h at RT. The reaction can also be performed in other solvents, such as DCM (dichloromethane) and MeCN (acetonitrile), affording the product in 62 and 60% yields (entries 3 and 4) respectively. The yield decreases when using protic MeOH (methanol) solvent, but interestingly there is still 40% of product (entry 5). Control reactions with NiCl₂(glyme) and NiBr₂(glyme) salts (entries 6 and 7; glyme=dimethoxyethane) as catalysts resulted in 1% and 4% desired product formation (entries 6 and 7). Using Ni(II) trifluoromethanesulfonate as catalyst resulted in 5% product formation after 24 h, (see entry 8). The control reaction in the absence of any nickel additive afforded 7% of trifluoromethylated product (see entry 9).

### Example 3 (General Procedure)

Inside the glovebox, a 20 mL vial equipped with a stirring bar was charged with substrate (1 equiv), 1-perfluoroalkyl-1,2-benziodoxol-3(1H)one (1-1.2 equiv, see below scheme) and (C₄F₈)Ni(MeCN)₂ (10 mol %) dissolved in anhydrous DMSO. The reaction mixture was vigorously stirred for 24-48 hours. After reaction completion, the reaction mixture was transferred to a separatory funnel outside the glovebox and mixed with ethyl acetate and saturated aqueous NaCl. After the first extraction, the organic layer was separated, and the remaining water layer was again extracted with ethyl acetate. The organic layers were combined and dried over magnesium sulfate. Further the reaction mixture was concentrated under vacuum. The reaction mixture was then purified by flash column chromatography on silica gel or HPLC method afforded the desired product.

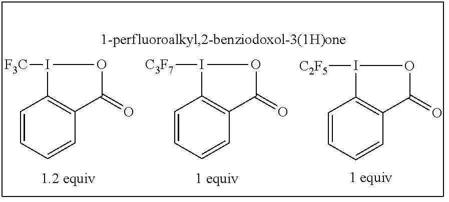

### Example 4

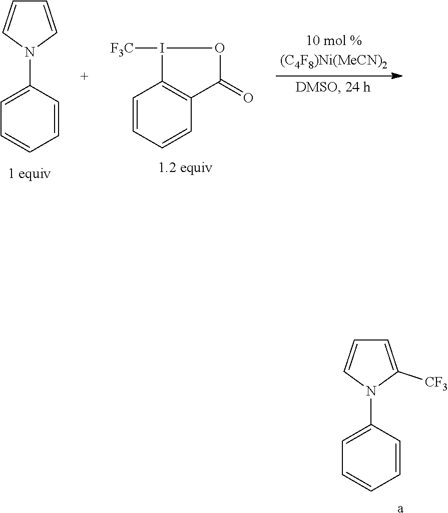

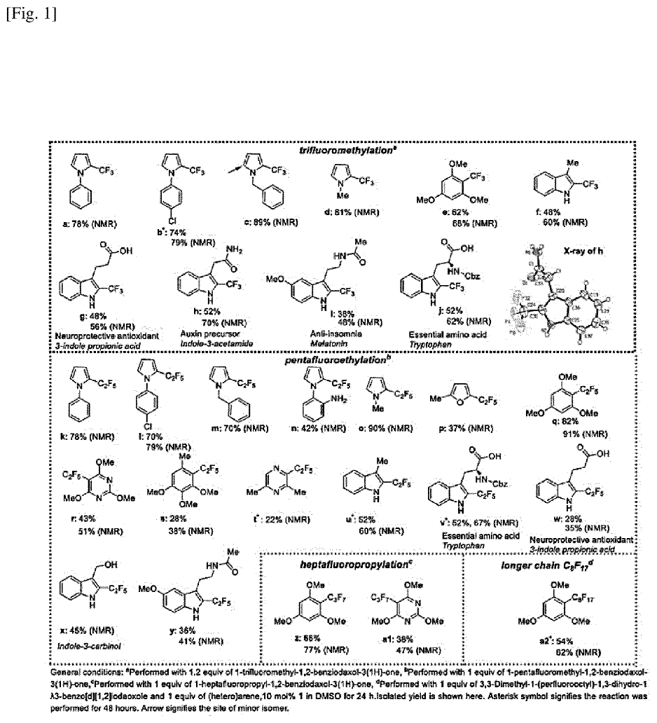

Inside the glovebox, a 20 mL vial equipped with a magnetic stirring bar was charged with 1-phenylpyrrole (10.0 mg, 0.069 mmol), 1-trifluoromethyl-1,2-benziodoxol-3(1H)-one (66.2 mg, 0.082 mmol) and (C₄F₈)Ni(MeCN)₂ (2.2 mg, 6.9 μmol). Then anhydrous DMSO (1 mL) was added. The reaction mixture was stirred vigorously at room temperature for 24 hours. After reaction completion, α,α,α-trifluorotoluene (8.5 μL, 0.069 mmol) was added as a standard and the yield was determined by ¹⁹F NMR spectroscopy. The yield was 78% (FIG. 1).

### Example 5

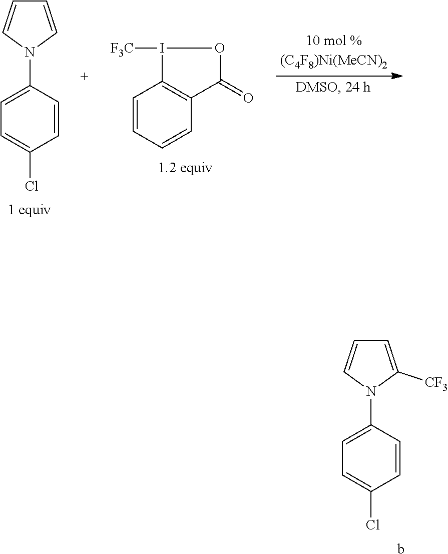

The general procedure was followed using 1-(4-Chlorophenyl)-1H-pyrrole (100 mg, 0.56 mmol), 1-trifluoromethyl-1,2-benziodoxol-3(1H)one (534 mg, 0.67 mmol), (C₄F₈)Ni(MeCN)₂ (19 mg, 0.056 mmol) in anhydrous DMSO (3 mL). The reaction mixture was vigorously stirred for 48 hours. Purification by flash column chromatography (mobile phase: hexane/ethyl acetate (95:5) gave b as colorless solid (103 mg, 74% yield, FIG. 1).

¹H NMR (500 MHz, CDCl₃, 23 °C): δ 7.44-7.42 (m, 2H), 7.33-7.31 (br.m, 2H), 6.86-6.85 (m, 1H), 6.75-6.73 (m, 1H), 6.29-6.28 (m, 1H).

¹³C NMR (101 MHz, CDCl₃, 23 °C): δ 137.7, 134.6, 129.4, 127.9, 127.3 (q, J=1.7 Hz), 122.4 (q, J=38.1 Hz), 120.6 (q, J=268.1 Hz), 113.2 (q, J=3.4 Hz), 108.7.

¹⁹F NMR (565 MHz, CDCl₃, 23 °C): δ −55.8 (3F) HRMS (ESI) calculated for [M*H]⁺, C₁₁H₇F₃N₁Cl₁: m/z 246.0292; found, 246.0294.

### Example 6

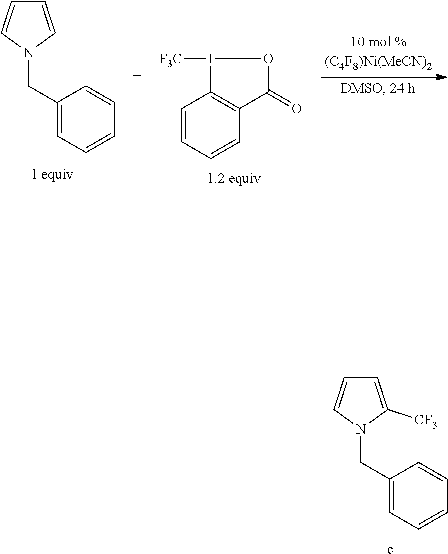

A 20 mL vial equipped with a magnetic stirring bar was charged with N-benzylpyrrole (7.7 μL, 0.05 mmol), 1-trifluoromethyl-1,2-benziodoxol-3(1H)-one (47.4 mg, 0.06 mmol) and (C₄F₈)Ni(MeCN)₂ (1.7 mg, 0.005 mmol), and then dissolved in anhydrous DMSO (1 mL). The reaction mixture was stirred at room temperature for 24 hours. After reaction completion, α,α,α-trifluorotoluene (6.1 L, 0.05 mmol) was added as a standard and the yield was determined by ¹⁹F NMR spectroscopy. The yield was 89% (FIG. 1).

### Example 7

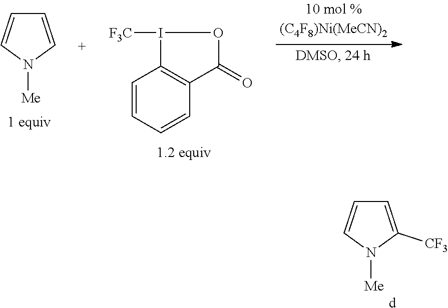

A 20 mL vial equipped with a magnetic stirring bar was charged with N-Methyl pyrrole (8.8 L, 0.10 mmol), 1-trifluoromethyl-1,2-benziodoxol-3(1H)-one (94.8 mg, 0.12 mmol) and (C₄F₈)Ni(MeCN)₂ (3.4 mg, 0.01 mmol), were dissolved in anhydrous DMSO (1 mL). Further the reaction mixture was stirred at room temperature for 24 hours. After reaction completion, α,α,α-trifluorotoluene (12.2 μL, 0.1 mmol) was added as a standard and the yield was determined by ¹⁹F NMR spectroscopy. The yield was 81% (FIG. 1).

### Example 8

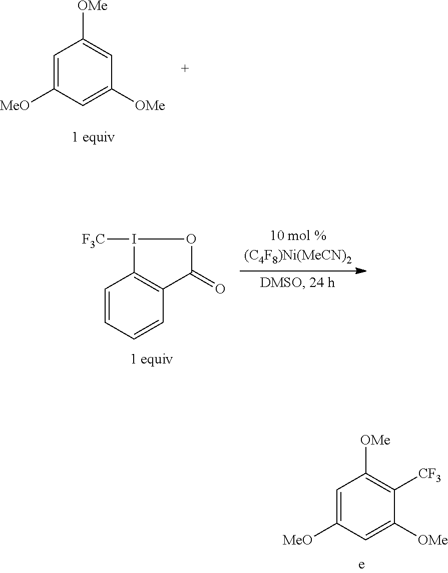 [SMILES: COc1cc(OC)cc(OC)c1 ; O=C1OI(C(F)(F)F)c2ccccc21 ; COc1cc(OC)c(C(F)(F)F)c(OC)c1]

The general procedure was followed using Trimethoxy benzene (100 mg, 0.59 mmol), 1-trifluoromethyl-1,2-benziodoxol-3(1H)one (564 mg, 0.70 mmol), (C₄F₈)Ni(MeCN)₂ (20.2 mg, 0.059 mmol) in anhydrous DMSO (3 mL). The reaction mixture was vigorously stirred for 24 hours. Purification by flash column chromatography was completed (mobile phase: hexane/ethyl acetate (95:5) as colorless solid (88 mg, 62% yield, FIG. 1).

¹H NMR (400 MHz, CDCl₃, 23 °C): δ 6.13 (s, 2H), 3.84 (s, 9H).

¹³C NMR (101 MHz, CDCl₃, 23 °C): δ 163.6, 160.6, 124.5 (q, J=273.1 Hz), 100.7-100.4 (m), 91.4, 56.4, 55.5.

¹⁹F NMR (376 MHz, CDCl₃, 23 °C): δ −54.0 (3F).

HRMS (ESI) calculated for [M*H]⁺, C₁₀H₁₁F₃O₃: m/z 237.0733; found, 237.0753.

### Example 9

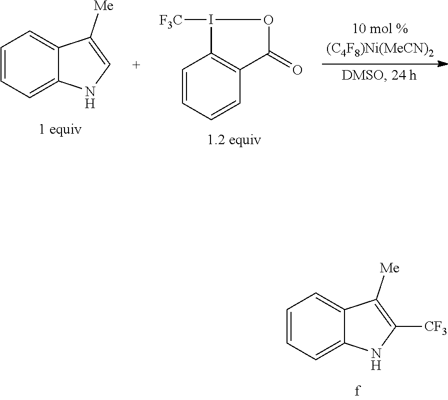 [SMILES: Cc1c[nH]c2ccccc12 ; O=C1OI(C(F)(F)F)c2ccccc21 ; Cc1c(C(F)(F)F)[nH]c2ccccc12]

The general procedure was followed using 3-methylindole (50 mg, 0.38 mmol), 1-trifluoromethyl-1,2-benziodoxol-3(1H)one (362 mg, 0.45 mmol), (C₄F₈)Ni(MeCN)₂ (13 mg, 0.038 mmol) in anhydrous DMSO (1.5 mL). The reaction mixture was vigorously stirred for 24 hours. Purification by flash column chromatography (mobile phase: hexane/ethyl acetate (95:5) as colorless solid (37 mg, 48% yield, FIG. 1).

¹H NMR (600 MHz, CDCl₃, 23 °C): δ 8.18 (br s, 1H), 7.64 (d, J=8.1 Hz, 1H), 7.38 (d, J=8.1 Hz, 1H), 7.32 (t, J=7.6 Hz, 1H), 7.19 (t, J=7. Hz, 1H), 2.44 (m, 3H).

¹³C NMR (151 MHz, CDCl₃, 23 °C): δ 135.3, 128.2, 124.9, 123.1 (q, J=268.4 Hz, CF3), 121.6 (q, J=36.4 Hz), 120.5, 120.2, 114.2 (q, J=113.0 Hz), 111.7, 8.5.

¹⁹F NMR (565 MHz, CDCl₃, 23 °C): δ −58.5 (3F).

HRMS (ESI) calculated for [M*H]⁺, C₁₀H₉F₃N: m/z 200.0682; found, 200.0680.

### Example 10

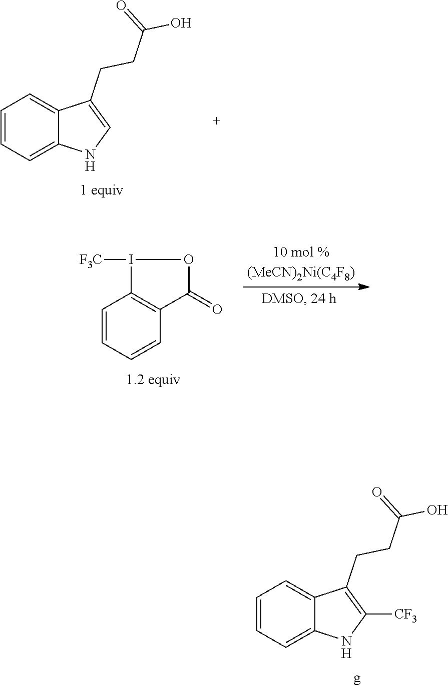 [SMILES: O=C(O)CCc1c[nH]c2ccccc12 ; O=C1OI(C(F)(F)F)c2ccccc21 ; O=C(O)CCc1c(C(F)(F)F)[nH]c2ccccc12]

The general procedure was followed using 3-indolepropionic acid (100 mg, 0.52 mmol), 1-trifluoromethyl-1,2-benziodoxol-3(1H)one (501 mg, 0.62 mmol), (C₄F₈)Ni(MeCN)₂ (18 mg, 0.052 mmol) in anhydrous DMSO (3 mL). The reaction mixture was vigorously stirred for 24 hours. Purification by HPLC afforded g as a pale yellow solid (65 mg, 48% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (400 MHz, CD₃OD, 23 °C): δ 7.65 (d, J=8.1 Hz, 1H), 7.41 (d, J=8.1 Hz, 1H), 7.25 (d, J=7.5 Hz, 1H), 7.10 (d, J=7.5 Hz, 1H), 3.19 (t, J=7.6 Hz, 2H), 2.59 (t, J=7.6 Hz, 2H). (O—H and N—H protons could not observed)

¹³C NMR (101 MHz, CDCl₃, 23 °C): δ 176.6, 137.4, 128.0, 125.3, 125.1 (q, J=271.1 Hz), 122.7 (q, J=36.8 Hz), 121.1, 120.7, 116.9 (m), 113.1, 36.6, 20.5.

¹⁹F NMR (376 MHz, CDCl₃, 23 °C): δ −58.3 (3F).

HRMS (ESI) calculated for [M*Na]⁺, C₁₂H₁₀F₃NO₂: m/z 280.0556; found, 280.0564.

### Example 11

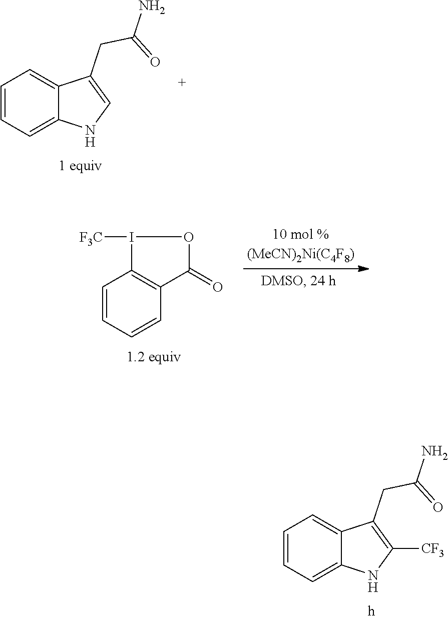 [SMILES: NC(=O)Cc1c[nH]c2ccccc12 ; O=C1OI(C(F)(F)F)c2ccccc21 ; NC(=O)Cc1c(C(F)(F)F)[nH]c2ccccc12]

The general procedure was followed using Indole-3-acetamide (50 mg, 0.28 mmol), 1-trifluoromethyl-1,2-benziodoxol-3(1H)one (272 mg, 0.33 mmol), (C₄F₈)Ni(MeCN)₂ (9.7 mg, 0.028 mmol) in anhydrous DMSO (2 mL). The reaction mixture was vigorously stirred for 24 hours. Purification by HPLC afforded h as a faint yellow white solid (44 mg, 52% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (500 MHz, CDCl₃, 23 °C): δ 9.21 (br.s, 1H), 7.63 (d, J=8.2 Hz, 1H), 7.42 (d, J=8.2 Hz, 1H), 7.34 (t, J=7.5 Hz, 1H), 7.22 (t, J=7.5 Hz, 1H), 6.59 (br.s, 1H), 5.73 (br.s, 1H), 3.90 (s, 2H).

¹³C NMR (126 MHz, CDCl₃, 23 °C): δ 175.1, 135.5, 126.9, 125.6, 123.4 (q, J=37.1 Hz), 122.1 (q, J=267.2 Hz), 121.7, 120.0, 112.3, 109.9, 29.8.

¹⁹F NMR (376 MHz, CDCl₃, 23 °C): δ −58.2 (3F).

HRMS (ESI) calculated for [M*H]⁺, C₁₁H₉F₃N₂O: m/z 243.0740; found, 243.0766.

### Example 12

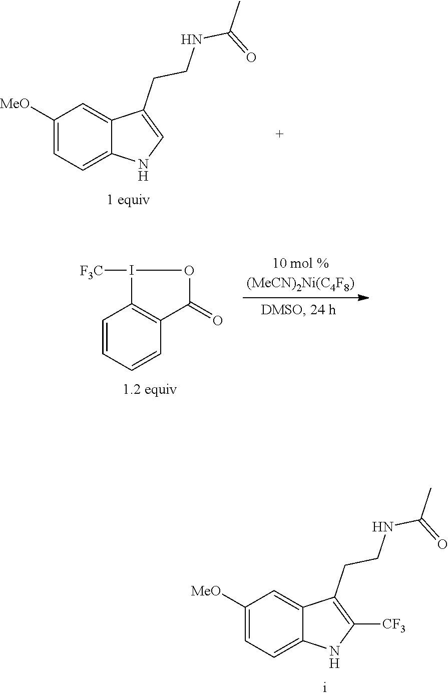 [SMILES: COc1ccc2[nH]cc(CCNC(C)=O)c2c1 ; O=C1OI(C(F)(F)F)c2ccccc21 ; COc1ccc2[nH]c(C(F)(F)F)c(CCNC(C)=O)c2c1]

The general procedure was followed using Melatonin (50 mg, 0.21 mmol), 1-trifluoromethyl-1,2-benziodoxol-3(1H)one (204.1 mg, 0.25 mmol), (C₄F₈)Ni(MeCN)₂ (7.3 mg, 0.021 mmol) in anhydrous DMSO (3 mL). The reaction mixture was vigorously stirred for 24 hours. Purification by HPLC afforded i as a faint colorless oil (25 mg, 38% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (500 MHz, CDCl₃, 23 °C): δ 10.06 (br.s, 1H), 8.47 (br s, 1H), 7.22 (d, J=8.8 Hz, 1H), 6.97 (d, J=2.3 Hz, 1H), 6.91 (dd, J=8.9, 2.3 Hz, 1H), 3.78 (s, 3H), 3.49 (m, 2H), 3.01 (t, J=6.7 Hz, 2H), 1.95 (s, 3H).

¹³C NMR (151 MHz, CDCl₃, 23 °C): δ 173.5, 155, 130.5, 127.7, 122.9 (q, J=36.6 Hz), 116.4, 113.9 (q, J=2.6 Hz), 113.1, 100.3, 55.9, 40.6, 23.6, 22.4.

¹⁹F NMR (565 MHz, CDCl₃, 23 °C): δ −58.3 (3F).

HRMS (ESI) calculated for [M*H]⁺, C₁₄H₁₆F₃N₂O₂: m/z 301.1158; found, 301.1180.

### Example 13

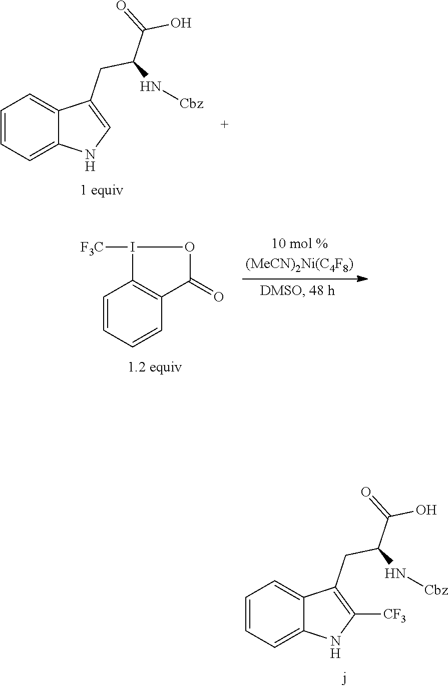 [SMILES: O=C(N[C@@H](Cc1c[nH]c2ccccc12)C(=O)O)OCc1ccccc1 ; O=C1OI(C(F)(F)F)c2ccccc21 ; O=C(N[C@@H](Cc1c(C(F)(F)F)[nH]c2ccccc12)C(=O)O)OCc1ccccc1]

The general procedure was followed using N-Cbz-L-Tryptophan (100 mg, 0.29 mmol), 1-trifluoromethyl-1,2-benziodoxol-3(1H)one (281 mg, 0.35 mmol), (C₄F₈)Ni(MeCN)₂ (10 mg, 0.029 mmol) in anhydrous DMSO (3 mL). The reaction mixture was vigorously stirred for 24 hours. Purification by HPLC afforded j as a colorless solid (63 mg, 52% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (400 MHz, CD₃OD, 23 °C): δ 11.47 (br.s, 1H), 7.73 (d, J=8.1 Hz, 1H), 7.41 (d, J=8.1 Hz, 1H), 7.25 (m, 8H), 7.09 (t, J=7.5 Hz, 1H), 4.49 (dd, J=8.4, 5.8 Hz, 1H), 3.49 (dd, J=14.6, 5.1 Hz, 1H), 3.27 (m, 1H). (Two protons overlap with water peak)

¹³C NMR (101 MHz, CD₃OD, 23 °C): δ 174.9, 158.2, 138.0, 137.3, 129.4, 128.9, 128.6, 125.3, 123.7 (q, J=36.2 Hz), 123.4 (q, J=261.2 Hz), 121.2, 121.1, 113.6, 113.0, 67.5, 56.5, 27.8.

¹⁹F NMR (376 MHz, CD₃OD, 23 °C): δ −59 (3F).

HRMS (ESI) calculated for [M*Na]⁺, C₂₀H₁₇F₃N₂O₄: m/z 429.1033; found, 429.1005.

### Example 14

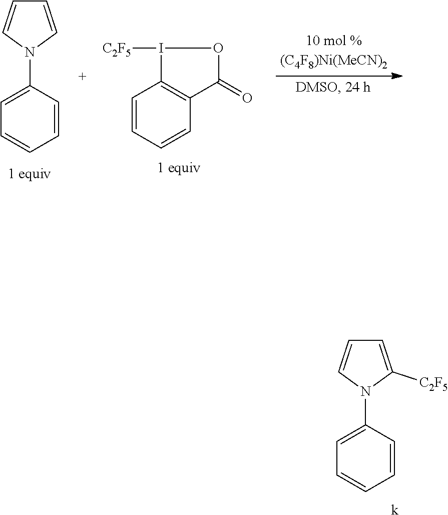

A 20 mL vial equipped with a magnetic stirring bar was charged with 1-phenylpyrrole (10.0 mg, 0.069 mmol), 1-pentafluoroethyl-1,2-benziodoxol-3(1H)-one (25.5 mg, 0.069 mmol) and (C₄F₈)Ni(MeCN)₂ (2.3 mg, 0.006 mmol), and then dissolved in anhydrous DMSO (1 mL). The reaction mixture was stirred at room temperature for 24 hours. After reaction completion, α,α,α-trifluorotoluene (8.5 μL, 0.069 mmol) was added as a standard and the yield was determined by ¹⁹F NMR spectroscopy. The yield was 78% (FIG. 1).

### Example 15

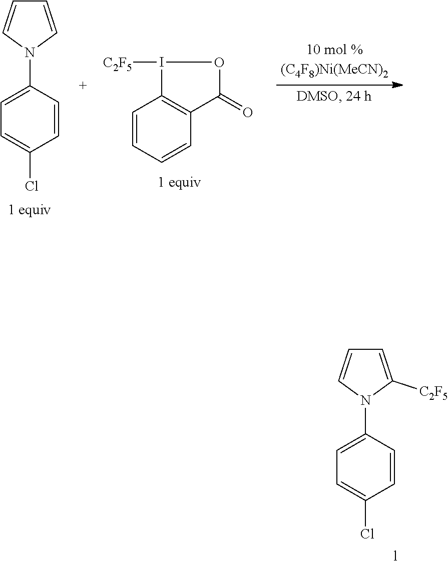

The general procedure was followed using 1-(4-Chlorophenyl)-1H-pyrrole (100 mg, 0.56 mmol), 1-pentafluoroethyl-1,2-benziodoxol-3(1H)one (206 mg, 0.56 mmol), (C₄F₈)Ni(MeCN)₂ (19 mg, 0.05 mmol) in anhydrous DMSO (3 mL). The reaction mixture was vigorously stirred for 24 hours. Purification by flash column chromatography (mobile phase: hexane/ethyl acetate (85:15) gave 1 as colorless solid (116 mg, 70% yield, FIG. 1).

¹H NMR (500 MHz, CDCl₃, 23 °C): δ 7.42-7.40 (m, 2H), 7.32-7.30 (br.m, 2H), 6.86 (br. s, 1H), 6.74 (br.s, 1H), 6.32 (m, 1H).

¹³C NMR (126, MHz, CDCl₃, 23 °C): δ 138.2, 134.8, 129.1, 128.7, 128.6, 122.6-117.5 (m), 120.1, 115.8-110.8 (m), 114.8 (m), 109.1.

¹⁹F NMR (376 MHz, CDCl₃, 23 °C): δ −83.0 (3F), −102.6 (2F).

HRMS (ESI) calculated for [M*H]⁺, C₁₂H₇F₅N₁Cl₁: m/z 296.0260; found, 296.0261.

### Example 16

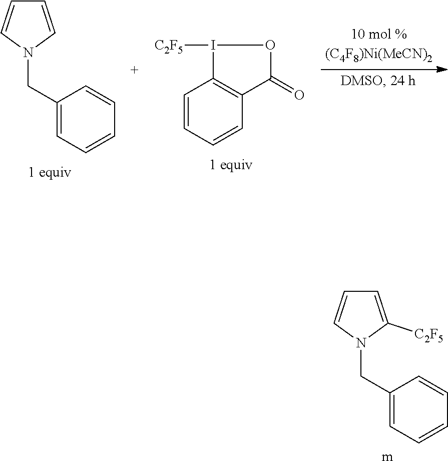

A 20 mL vial equipped with a magnetic stirring bar was charged with N-benzylpyrrole (15.4 μL, 0.1 mmol), 1-pentafluoroethyl-1,2-benziodoxol-3(1H)-one (36.6 mg, 0.1 mmol) and (C₄F₈)Ni(MeCN)₂ (3.4 mg, 0.01 mmol), and then dissolved in anhydrous DMSO (1 mL). The reaction mixture was stirred at room temperature for 24 hours. After reaction completion, α,α,α-trifluorotoluene (12.2 μL, 0.1 mmol) was added as a standard and the yield was determined by ¹⁹F NMR spectroscopy. The yield was 70% (FIG. 1).

### Example 17

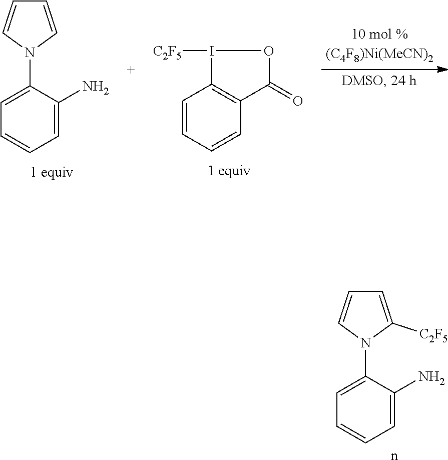

A 20 mL vial equipped with a magnetic stirring bar was charged with 1-(2-Aminophenyl)pyrrole (10 mg, 0.063 mmol), 1-pentafluoroethyl-1,2-benziodoxol-3(1H)-one (23.1 mg, 0.063 mmol) and (C₄F₈)Ni(MeCN)₂ (2.1 mg, 0.006 mmol), and then dissolved in anhydrous DMSO (1 mL). The reaction mixture was stirred at room temperature for 24 hours. After reaction completion, α,α,α-trifluorotoluene (7.7 μL, 0.063 mmol) was added as a standard and the yield was determined by ¹⁹F NMR spectroscopy. The yield was 42% (FIG. 1).

### Example 18

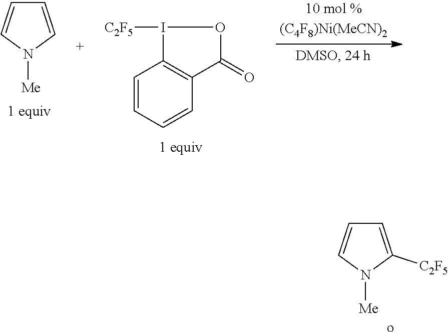

A 20 mL vial equipped with a magnetic stirring bar was charged with N-Methyl pyrrole (8.8 L, 0.10 mmol), 1-pentafluoroethyl-1,2-benziodoxol-3(1H)-one (36.6 mg, 0.10 mmol) and (C₄F₈)Ni(MeCN)₂ (3.4 mg, 0.01 mmol), were dissolved in anhydrous DMSO (1 mL). Further the reaction mixture was stirred at room temperature for 24 hours. After reaction completion, α,α,α-trifluorotoluene (12.2 μL, 0.1 mmol) was added as a standard and the yield was determined by ¹⁹F NMR spectroscopy. The yield was 90% (FIG. 1).

### Example 19

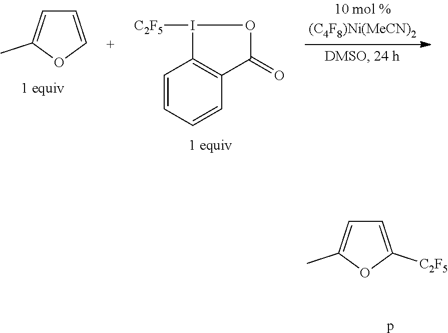

A 20 mL vial equipped with a magnetic stirring bar was charged with 2-Methylfuran (4.4 μL, 0.050 mmol), 1-pentafluoroethyl-1,2-benziodoxol-3(1H)-one (18.3 mg, 0.050 mmol) and (C₄F₈)Ni(MeCN)₂ (1.7 mg, 0.005 mmol), which were dissolved in anhydrous DMSO (1 mL). The reaction mixture was stirred at room temperature for 24 hours. After reaction completion, α,α,α-trifluorotoluene (6.1 L, 0.05 mmol) was added as a standard and the yield was determined by ¹⁹F NMR spectroscopy. The yield was 37% (FIG. 1).

### Example 20

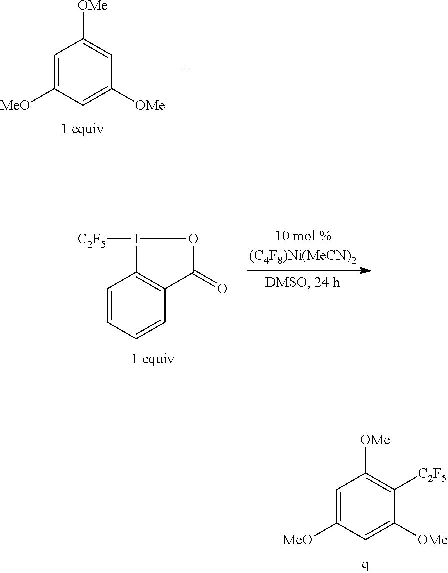

The general procedure was followed using Trimethoxy benzene (100 mg, 0.59 mmol), 1-pentafluoroethyl-1,2-benziodoxol-3(1H)one (217.6 mg, 0.59 mmol), (C₄F₈)Ni(MeCN)₂ (20.2 mg, 0.059 mmol) in anhydrous DMSO (3 mL). The reaction mixture was vigorously stirred for 24 hours. Purification by flash column chromatography (mobile phase: hexane/ethyl acetate (95:5) gave q as a colorless solid (140 mg, 82% yield, FIG. 1).

¹H NMR (600 MHz, CDCl₃, 23 °C): δ 6.15 (s, 2H), 3.83 (s, 3H), 3.81 (s, 6H).

¹³C NMR (151 MHz, CDCl₃, 23 °C): δ 163.9, 161.7, 123.1-116.9 (qt, J=285.1 Hz, 38.9 Hz), 116.5-112.3 (tq, J=246.8 Hz, 40.2 Hz), 98.3 (t, J=21.8 Hz), 91.8, 56.4, 55.4.

¹⁹F NMR (565 MHz, CDCl₃, 23 °C): δ −85.0 (3F), −106.6 (2F).

HRMS (ESI) calculated for [M*Na]⁺, C₁₁H₁₁F₅O₃: m/z 309.0503; found, 309.0521.

### Example 21

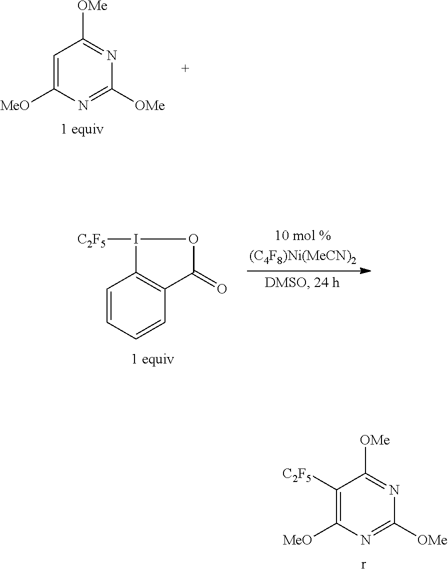

The general procedure was followed using 2,4,6-Trimethoxypyrimidine (100 mg, 0.58 mmol), 1-pentafluoroethyl-1,2-benziodoxol-3(1H)one (215 mg, 0.58 mmol), (C₄F₈)Ni(MeCN)₂ (20 mg, 0.059 mmol) in anhydrous DMSO (3 mL). The reaction mixture was vigorously stirred for 48 hours. Purification by flash column chromatography (mobile phase: hexane/ethyl acetate (90:10) gave r as a colorless solid (70 mg, 43% yield, FIG. 1).

¹H NMR (600 MHz, CDCl₃, 23 °C): δ 4.01 (s, 3H), 3.99 (s, 6H).

¹³C NMR (151 MHz, CDCl₃, 23 °C): δ 170.8, 165.4, 122.8-116.6 (tq, J=246.29 Hz, 40.2 Hz), 115.3-111.4 (qt, J=286.19 Hz, 39.7 Hz), 86.9 (t, J=25.5 Hz), 55.3, 55.0.

¹⁹F NMR (565 MHz, CDCl₃, 23 °C): δ −85.2 (3F), −109.6 (2F).

HRMS (ESI) calculated for [M*H]⁺, C₉H₉F₅N₂O₃: m/z 289.0606; found, 289.0605.

### Example 22

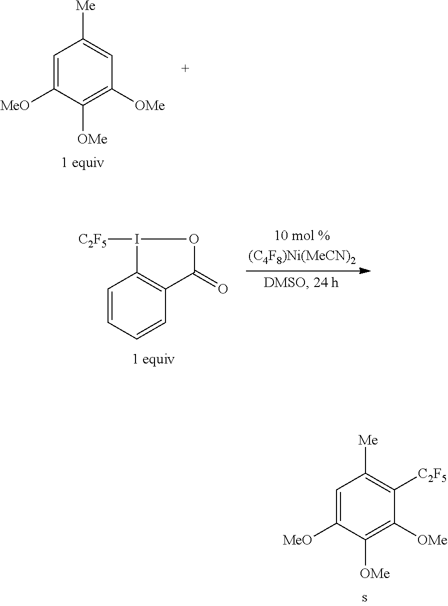

The general procedure was followed using 3,4,5-Trimethoxytoluene (50.5 μL, 0.30 mmol), 1-pentafluoroethyl-1,2-benziodoxol-3(1H)one (109.8 mg, 0.30 mmol), (C₄F₈)Ni(MeCN)₂ (10.2 mg, 0.03 mmol) in anhydrous DMSO (3 mL). The reaction mixture was vigorously stirred for 24 hours. Purification by flash column chromatography (mobile phase: hexane/ethyl acetate (90:10) gave s as colorless solid (28% yield, FIG. 1).

¹H NMR (600 MHz, CDCl₃, 23 °C): δ 6.52 (s, 1H), 3.89-3.88 (overlap of two singlets, 6H), 2.41 (t, J=4.8 Hz, 3H).

¹³C NMR (151 MHz, CDCl₃, 23 °C): δ 155.4, 154.6, 141.1, 135.1, 123.0-113.3 (m), 113.2 (t, J=21.1 Hz), 111.3, 62, 60.8, 56.0, 22.1 (t, J=6.6 Hz).

¹⁹F NMR (565 MHz, CDCl₃, 23 °C): δ −84.3 (3F), −105.4 (2F).

HRMS (ESI) calculated for [M*Na]⁺, C₁₂H₁₃F₅O₃: m/z 323.0677; found, 323.0688.

### Example 23

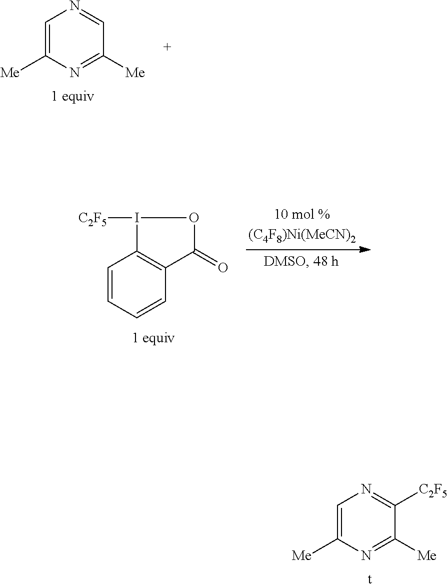

A 20 mL vial equipped with a magnetic stirring bar was charged with 2,6-Dimethylpyrazine (10.0 mg, 0.092 mmol), 1-pentafluoroethyl-1,2-benziodoxol-3(1H)-one (33.8 mg, 0.092 mmol) and (C₄F₈)Ni(MeCN)₂ (3.1 mg, 0.009 mmol), and then dissolved in anhydrous DMSO (1 mL). The reaction mixture was stirred at room temperature for 48 hours. After reaction completion, α,α,α-trifluorotoluene (11.3 μL, 0.092 mmol) was added as a standard and the yield was determined by ¹⁹F NMR spectroscopy. The yield was 22% (FIG. 1).

### Example 24

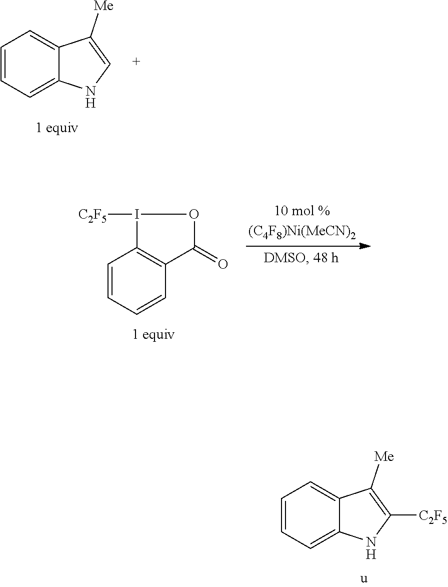

The general procedure was followed using 3-Methyl indole (50 mg, 0.38 mmol), 1-pentafluoroethyl-1,2-benziodoxol-3(1H)-one (139.5 mg, 0.38 mmol), (C₄F₈)Ni(MeCN)₂ (12.9 mg, 0.038 mmol) in anhydrous DMSO (1.5 mL). The reaction mixture was vigorously stirred for 48 hours. Purification by preparatory TLC method (mobile phase: hexane/ethyl acetate (95:5) as colorless solid (50 mg, 52% yield, FIG. 1).

¹H NMR (600 MHz, CDCl₃, 23 °C): δ 8.16 (br s, 1H), 7.66 (d, J=8.1 Hz, 1H), 7.40 (d, J=8.1 Hz, 1H), 7.34 (t, J=7.7 Hz, 1H), 7.21 (t, J=7.7 Hz, 1H), 2.44 (s, 3H).

¹³C NMR (151 MHz, CDCl₃, 23 °C): δ 135.9, 128.5, 125.0, 122.5-118.2 (m), 120.5, 120.1, 116.3 (t, J=3 Hz), 112.2 (tq, J=245.2 Hz), 111.6, 8.6.

¹⁹F NMR (565 MHz, CDCl₃, 23 °C): δ −84.7 (3F), −112.7 (2F).

HRMS (ESI) calculated for [M*H]⁺, C₁₁H₉F₅N₁: m/z 250.0650; found, 250.0669.

### Example 25

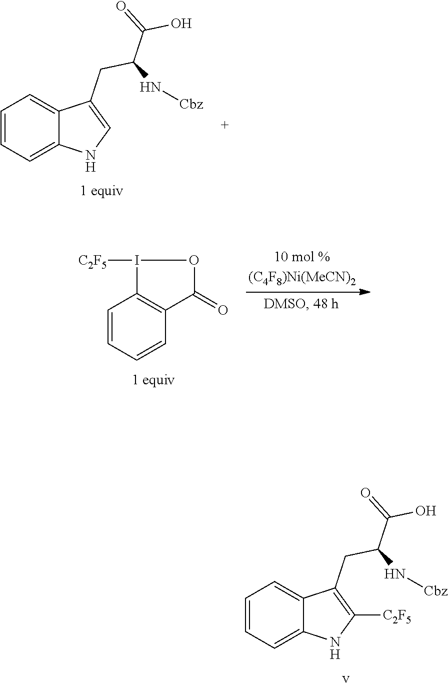

The general procedure was followed using N-Cbz-L-Tryptophan (100.2 mg, 0.29 mmol), 1-pentafluoroethyl-1,2-benziodoxol-3(1H)-one (108.3 mg, 0.29 mmol), (C₄F₈)Ni(MeCN)₂ (10 mg, 0.029 mmol) in anhydrous DMSO (3 mL). The reaction mixture was vigorously stirred for 48 hours. Purification by flash column chromatography (mobile phase: hexane/ethyl acetate (85:15) as colorless solid (70.1 mg, 52% yield, FIG. 1).

¹H NMR (600 MHz, CDCl₃, 23 °C): δ 8.46 (br.s, 1H), 7.74 (d, J=8 Hz, 1H), 7.38 (d, J=8 Hz, 1H), 7.34-7.28 (m, 6H), 7.26-7.24 (m, 2H), 7.17 (t, J=8 Hz, 1H), 5.35 (d, J=8 Hz, 1H) 5.02-4.98 (m, 1H), 4.74-4.70 (m, 1H), 3.45-3.42 (m, 1H), 3.31-3.27 (m, 1H).

¹³C NMR (151 MHz, CDCl₃, 23 °C): δ 175.8, 156.0, 136.1, 128.6, 128.3, 128.2, 127.5, 125.4, 121.3, 121.2-120.0 (m, coupling constant could not be measured due to signal overlap), 120.2, 114.7, 112.0, 67.2, 54.6, 27.6.

¹⁹F NMR (376 MHz, CDCl₃ 23 °C): δ −82.5 (3F), −111.4 (2F).

HRMS (ESI) calculated for [M*H]⁺, C₂₁H₁₈F₅O₄N₂: m/z 457.1200; found, 457.1184.

### Example 26

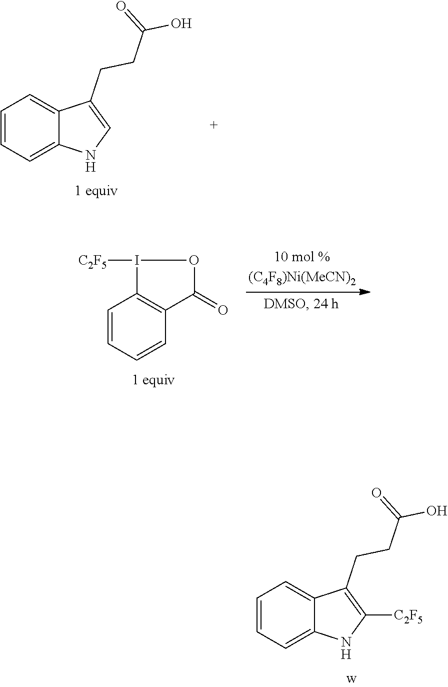

The general procedure was followed using Indole-3-propionic acid (100 mg, 0.52 mmol), 1-pentafluoroethyl-1,2-benziodoxol-3(1H)one (193.4 mg, 0.52 mmol), (C₄F₈)Ni(MeCN)₂ (18 mg, 0.052 mmol) in anhydrous DMSO (3 mL). The reaction mixture was vigorously stirred for 24 hours. Purification by HPLC afforded w as a faint yellow-colored solid (47 mg, 28% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (600 MHz, CDCl₃, 23 °C): δ 8.27 (br.s, 1H), 7.70 (d, J=8.1 Hz, 1H), 7.43 (d, J=8.3 Hz, 1H), 7.35 (t, J=7.6 Hz, 1H), 7.22 (t, J=7.5 Hz, 1H), 3.24 (m, 2H), 2.73 (m, 2H).

¹³C NMR (151 MHz, CDCl₃, 23 °C): δ 177.9, 136.1, 127.3, 125.3, 125.0-112.2 (spikes of fluorine signals), 121.0, 120.1, 119.5 (t, J=27.6 Hz), 118.7, 111.9, 35.1, 19.4.

¹⁹F NMR (565 MHz, CDCl₃, 23 °C): δ −84.6 (3F), −112.2 (2F).

HRMS (ESI) calculated for [M*Na]⁺, C₁₃H₁₀F₅O₂N₁Na₁: m/z 330.0524; found, 330.0538.

### Example 27

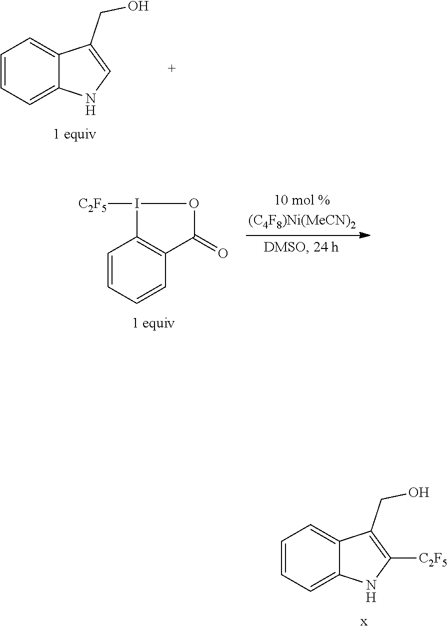

A 20 mL vial equipped with a magnetic stirring bar was charged with Indole-3-carbinol (10 mg, 0.0679 mmol), 1-pentafluoroethyl-1,2-benziodoxol-3(1H)-one (24.8 mg, 0.0679 mmol) and (C₄F₈)Ni(MeCN)₂ (2.3 mg, 0.0067 mmol), and then dissolved in anhydrous DMSO (1 mL). The reaction mixture was stirred at room temperature for 24 hours. After reaction completion, α,α,α-trifluorotoluene (8.3 μL, 0.0679 mmol) was added as a standard and the yield was determined by ¹⁹F NMR spectroscopy. The yield was 45% (FIG. 1).

### Example 28

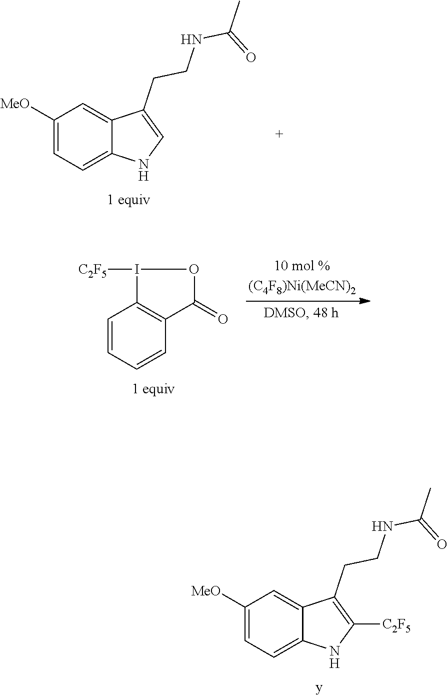

The general procedure was followed using Melatonin (100 mg, 0.43 mmol), 1-pentafluoroethyl-1,2-benziodoxol-3(1H)-one (157.6 mg, 0.43 mmol), (C₄F₈)Ni(MeCN)₂ (14.6 mg, 0.043 mmol) in anhydrous DMSO (3 mL). The reaction mixture was vigorously stirred for 48 hours. Purification by HPLC afforded y as a faint yellow-colored oil (54 mg, 36% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (600 MHz, CDCl₃, 23 °C): δ 8.42 (br s, 1H), 7.31 (d, J=8.8 Hz, 1H), 7.14 (br.s, 1H), 7 (dd, J=9.1, 1.8 Hz, 1H), 5.86 (br. s, 1H), 3.86 (s, 3H), 3.56 (m, 2H), 3.07 (t, J=6.8 Hz, 2H), 1.98 (s, 3H).

¹³C NMR (151 MHz, CDCl₃, 23 °C): δ 171.5, 155.1, 133.4-127.0 (m), 131.3, 128.2, 120.5-120.0 (m), 118.1 (m), 116.6-116.4 (m), 112.9, 110.8, 100.6, 55.9, 40.5, 24.2, 23.1.

¹⁹F NMR (376 MHz, CDCl₃, 23 °C): δ −82.5 (3F), −111.4 (2F).

HRMS (ESI) calculated for [M*H]⁺, C₁₅H₁₆F₅O₂N₂: m/z 351.1126; found, 351.1135.

### Example 29

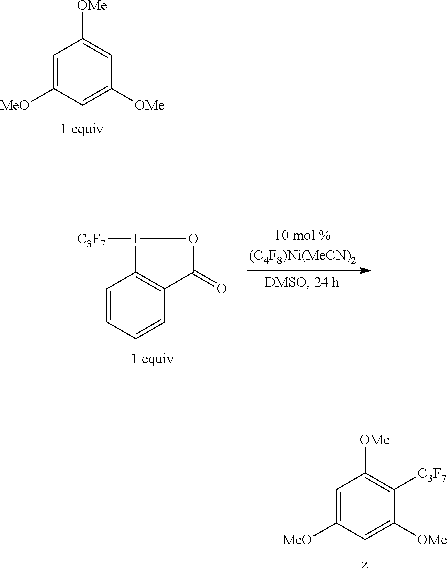

The general procedure was followed using 1,3,5-trimethoxybenzene (50 mg, 0.29 mmol), 1-heptafluoropropyl-1,2-benziodoxol-3(1H)one (123.6 mg, 0.29 mmol), (C₄F₈)Ni(MeCN)₂ (10 mg, 0.02 mmol) in anhydrous DMSO (1.5 mL). The reaction mixture was vigorously stirred for 24 hours. Purification by flash column chromatography (mobile phase: hexane/ethyl acetate (95:5) gave z as colorless solid (66 mg, 66% yield, FIG. 1).

¹H NMR (600 MHz, CDCl₃, 23 °C): δ 6.14 (s, 2H), 3.84 (s, 3H), 3.80 (s, 6H).

¹³C NMR (151 MHz, CDCl₃, 23 °C): δ 164.0, 161.8, 121.6-107.6 (m, fluorine splitting overlap), 98.4 (t, J=21.7 Hz), 91.8, 56.4, 55.5.

¹⁹F NMR (565 MHz, CDCl₃, 23 °C): δ −80.5 (3F), −103.4 (2F), −126.8 (2F).

HRMS (ESI) calculated for [M*H]₊, C₁₂H₁₁F₇O₃: m/z 337.0669; found, 337.0667.

### Example 30

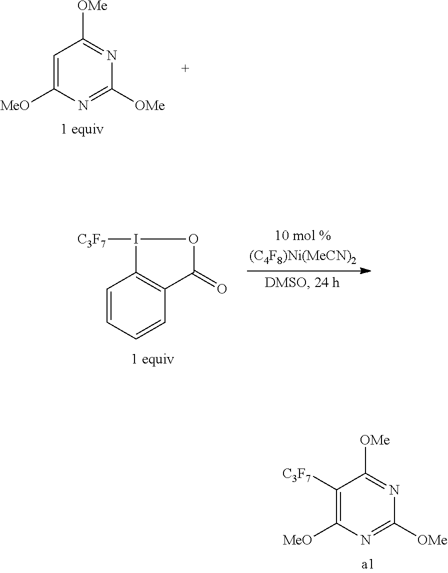

The general procedure was followed using 2,4,6-Trimethoxypyrimidine (50 mg, 0.29 mmol), 1-heptafluoropropyl-1,2-benziodoxol-3(1H)one (122.2 mg, 0.29 mmol), (C₄F₈)Ni(MeCN)₂ (10 mg, 0.02 mmol) in anhydrous DMSO (1.5 mL). The reaction mixture was vigorously stirred for 24 hours. Purification by flash column chromatography (mobile phase: hexane/ethyl acetate (90:10) gave al as colorless solid (38 mg, 38% yield, FIG. 1).

¹H NMR (600 MHz, CDCl₃, 23 °C): δ 4.01 (s, 3H), 3.98 (s, 6H).

¹³C NMR (151 MHz, CDCl₃, 23 °C): δ 170.9, 165.4, 121.3-113.4 (m, fluorine splitting overlap), 109.5 (qt, J=287.2, 41.5 Hz), 87.1 (t, J=25.5 Hz), 55.3, 55.

¹⁹F NMR (565 MHz, CDCl₃, 23 °C): δ −80.5 (3F), −106.5 (2F), −127.1 (2F).

HRMS (ESI) calculated for [M*H]⁺, C₁₀H₉F₇O₃N₂: m/z 339.0574; found, 339.0570.

### Example 31

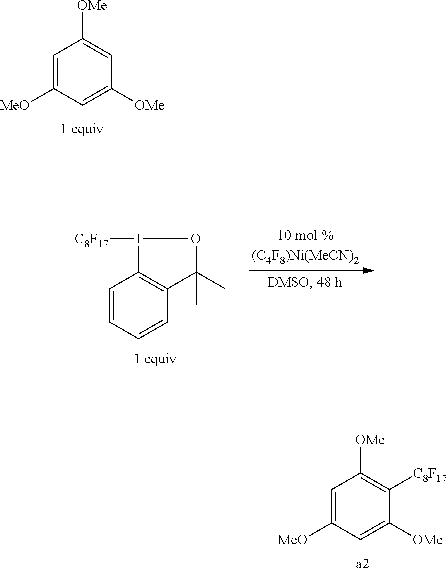

The general procedure was followed using 1,3,5-Trimethoxybenzene (100 mg, 0.59 mmol), 3,3-Dimethyl-1-(perfluorooctyl)-1,3-dihydro-1λ³-benzo[d][1,2]iodaoxole (404.3 mg, 0.59 mmol), (C₄F₈)Ni(MeCN)₂ (20.2 mg, 0.05 mmol) in anhydrous DMSO (3 mL). The reaction mixture was vigorously stirred for 48 hours. Purification by HPLC method afforded a2 as a colorless solid (190 mg, 54% yield, FIG. 1).

HPLC conditions: (A) MilliQ water and (B) Acetonitrile-Milli-Q water (90:10), both contained 0.1% formic acid. Gradient: 35% B for 0.0-1.0 min, 35-80% B for 1.0-6.0 min, hold 80% B for 6.0-9.0 min, equilibrate 80% B for 9.1-12.0 min.

¹H NMR (600 MHz, CDCl₃, 23 °C): δ 6.13 (s, 2H), 3.84 (s, 3H), 3.80 (s, 6H).

¹³C NMR (151 MHz, CDCl₃, 23 °C): δ 164.0, 161.9, 120.4-106.4 (m, fluorine splitting), 98.8 (t, J=21.8 Hz), 56.4, 55.5.

¹⁹F NMR (565 MHz, CDCl₃, 23 °C): δ −80.7 (3F), −102.6 (2F), −121.6-−122.7 (10F), −126.1 (2F).

HRMS (ESI) calculated for [M*H]⁺, C₁₇H₁₁F₁₇O₃: m/z 587.0510; found, 587.0347.

### Example 32

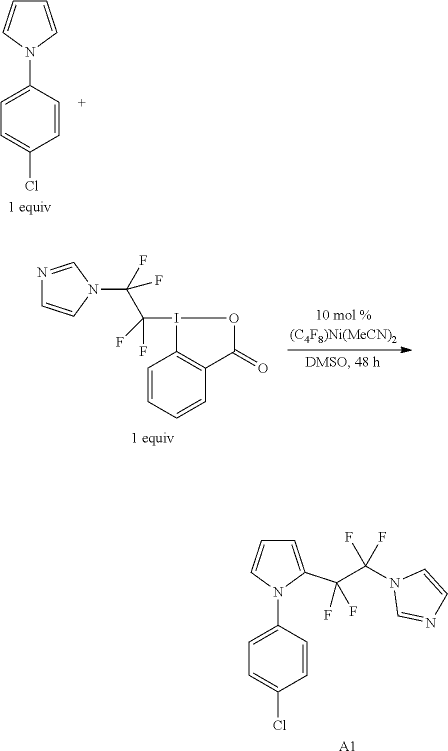 [SMILES: Clc1ccc(-n2cccc2)cc1 ; O=C1OI(C(F)(F)C(F)(F)n2ccnc2)c2ccccc21 ; FC(F)(c1cccn1-c1ccc(Cl)cc1)C(F)(F)n1ccnc1 ; "A1"]

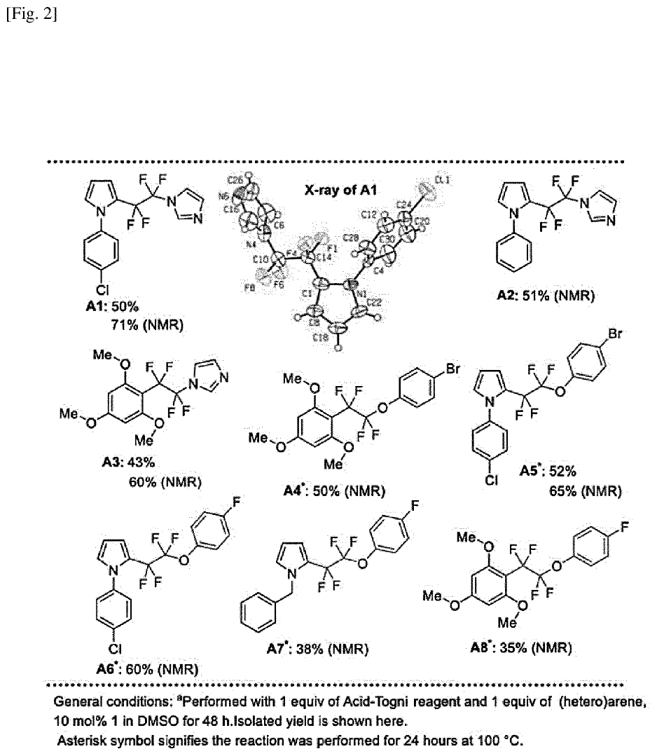

Inside the glovebox, a 20 mL vial equipped with a stirring bar was charged with 1-(4-Chlorophenyl)-1H-pyrrole (50 mg, 0.28 mmol), 1-(1,1,2,2-Tetrafluoro-2-(1H-imidazol-1-yl)ethyl)-1λ³-benzo[d][1,2]iodaoxol-3(1H)-one (116.5 mg, 0.28 mmol), and (C₄F₈)Ni(MeCN)₂ (9.5 mg, 0.02 mmol) dissolved in anhydrous DMSO (2 mL). The reaction mixture was vigorously stirred for 48 hours. After reaction completion, the reaction mixture was transferred to a separatory funnel outside the glovebox and mixed with ethyl acetate and saturated aqueous NaCl. After the first extraction, the organic layer was separated, and the remaining water layer was again extracted with ethyl acetate. The organic layers were combined and dried over magnesium sulfate. Further the reaction mixture was concentrated under vacuum. The reaction mixture was then purified by flash column chromatography to obtain A1 as a colorless solid (48 mg, 50% yield, FIG. 2).

¹H NMR (400 MHz, CDCl₃, 23 °C): δ 7.97 (dd, J=26.9, 7.4 Hz, 1H), 7.70 (br.s, 1H), 7.39-7.37 (m, 2H), 7.20-7.15 (m, 2H), 6.99 (s, 1H), 6.83 (br.s, 1H), 6.64 (br.s, 1H), 6.32 (br.s, 1H).

¹³C NMR (151 MHz, CDCl₃, 23 °C): δ 169.2, 141.5, 138, 135.7, 135.4-135.2 (m), 134.8, 132.6, 131.4, 129.7-128 (m), 119.5 (t, J=27.8 Hz), 117.0, 115.4, 109.4, 94.4.

¹⁹F NMR (565 MHz, CDCl₃, 23 °C): δ −94.6 (2F), −102.2 (2F).

HRMS (ESI) calculated for [M*H]⁺, C₁₅H₁₀F₄N₃Cl: m/z 344.0572; found, 344.0558.

### Example 33

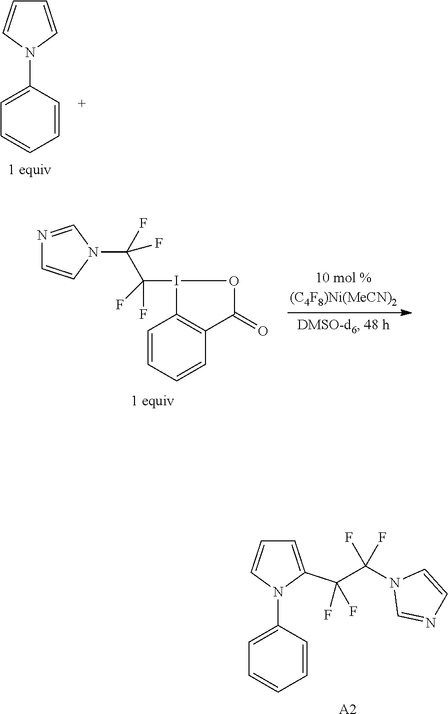 [SMILES: c1ccc(-n2cccc2)cc1 ; O=C1OI(C(F)(F)C(F)(F)n2ccnc2)c2ccccc21 ; FC(F)(c1cccn1-c1ccccc1)C(F)(F)n1ccnc1 ; "A2"]

Inside the glovebox, a 20 mL vial equipped with a magnetic stirring bar was charged with 1-Phenylpyrrole (10 mg, 0.0698 mmol), 1-(1,1,2,2-Tetrafluoro-2-(1H-imidazol-1-yl)ethyl)-1λ³-benzo[d][1,2]iodaoxol-3(1H)-one (28.9 mg, 0.0698 mmol) and (C₄F₈)Ni(MeCN)₂ (2.3 mg, 0.0067 mmol), and then dissolved in anhydrous deuterated DMSO (1 mL). The reaction mixture was stirred at room temperature for 48 hours. After reaction completion, α,α,α-trifluorotoluene (8.5 μL, 0.0698 mmol) was added as a standard and the yield was determined by ¹⁹F NMR spectroscopy. The yield was 51% (FIG. 2).

### Example 34

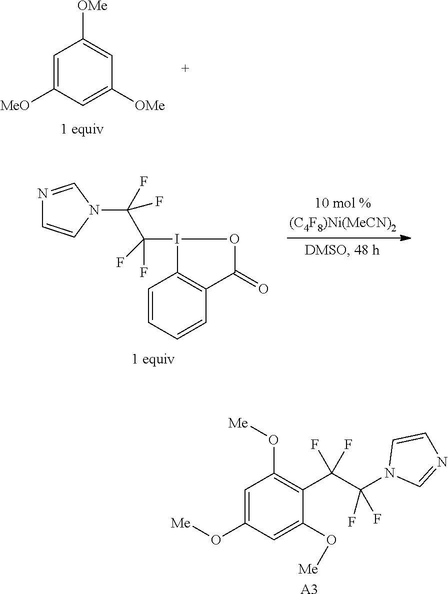

Inside the glovebox, a 20 mL vial equipped with a stirring bar was charged with 1,3,5-Trimethoxybenzene (50 mg, 0.29 mmol), 1-(1,1,2,2-Tetrafluoro-2-(1H-imidazol-1-yl)ethyl)-1λ³-benzo[d][1,2]iodaoxol-3(1H)-one (123.1 mg, 0.29 mmol) and (C₄F₈)Ni(MeCN)₂ (10.1 mg, 0.029 mmol) dissolved in anhydrous DMSO (2 mL). The reaction mixture was vigorously stirred for 48 hours. After reaction completion, the reaction mixture was transferred to a separatory funnel outside the glovebox and mixed with ethyl acetate and saturated aqueous NaCl. After the first extraction, the organic layer was separated, and remaining water layer was again extracted with ethyl acetate. The organic layers were combined and dried over magnesium sulfate. Further, the reaction mixture was concentrated under vacuum. The reaction mixture was then purified by flash column chromatography to obtain A3 as a colorless solid (42.8 mg, 43% yield, FIG. 2).

¹H NMR (400 MHz, CDCl₃, 23 °C): δ 7.62 (br.s, 1H), 7.07-7.02 (br.d, 2H), 6.05 (s, 2H), 3.81 (s, 3H), 3.64 (s, 6H).

¹³C NMR (151 MHz, CDCl₃, 23 °C): δ 164.0, 161.6, 141.3, 132.3-128.0 (m), 117.9-115.9 (m), 97.3 (t, J=21.6 Hz), 91.2, 56.1, 55.5.

¹⁹F NMR (565 MHz, CDCl₃, 23 °C): δ −98.2 (2F), −105.6 (2F).

HRMS (ESI) calculated for [M*H]⁺, C₁₄H₁₄F₄O₃N₂: m/z 335.1000; found, 335.1013.

### Example 35

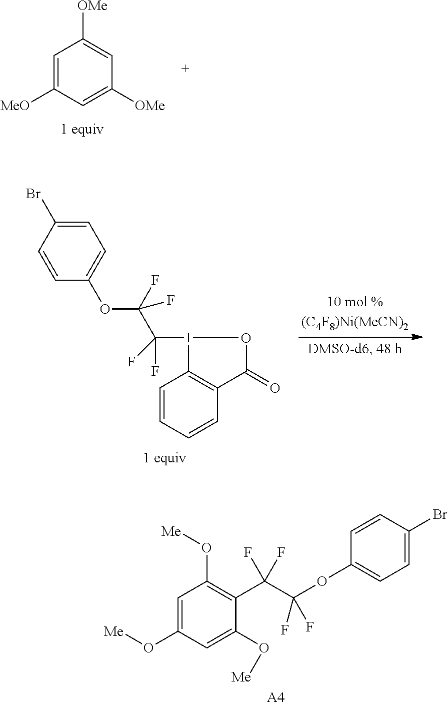

A septum vial equipped with a magnetic stirring bar was charged with 1,3,5-Trimethoxybenzene (10 mg, 0.059 mmol), 1-(2-(4-Bromophenoxy)-1,1,2,2-tetrafluoroethyl)-1λ³-benzo[d][1,2]iodaoxol-3(1H)-one (30.8 mg, 0.059 mmol) and (C₄F₈)Ni(MeCN)₂ (2 mg, 0.0059 mmol), and then dissolved in anhydrous deuterated DMSO (1 mL). The reaction mixture was transferred to an oil bath at 100 °C for 24 hours. After reaction completion, α,α,α-trifluorotoluene (7.3 μL, 0.059 mmol) was added as a standard and the yield was determined by ¹⁹F NMR spectroscopy. The yield was 50% (FIG. 2).

### Example 36

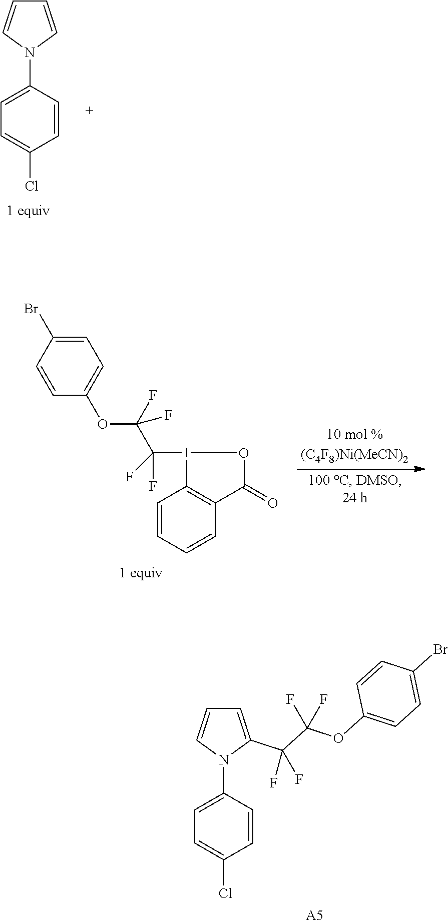 [SMILES: Clc1ccc(-n2cccc2)cc1 ; O=C1OI(C(F)(F)C(F)(F)Oc2ccc(Br)cc2)c2ccccc21 ; FC(F)(Oc1ccc(Br)cc1)C(F)(F)c1cccn1-c1ccc(Cl)cc1 ; "A5"]

Inside the glovebox, a 20 mL vial equipped with a stirring bar was charged with 1-(4-Chlorophenyl)-1H-pyrrole (50 mg, 0.28 mmol), 1-(2-(4-Bromophenoxy)-1,1,2,2-tetrafluoroethyl)-1λ³-benzo[d][1,2]iodaoxol-3(1H)-one (146 mg, 0.28 mmol) and (C₄F₈)Ni(MeCN)₂ (9.5 mg, 0.02 mmol) dissolved in anhydrous DMSO (2 mL). The reaction mixture was then transferred to an oil bath at 100 °C for 24 hours. After reaction completion, the reaction mixture was transferred to a separatory funnel outside the glovebox and mixed with ethyl acetate and saturated aqueous NaCl. After the first extraction, the organic layer was separated, and the remaining water layer was again extracted with ethyl acetate. The organic layers were combined and dried over magnesium sulfate. Further the reaction mixture was concentrated under vacuum. The reaction mixture was then purified by flash column chromatography to obtain A5 as a yellow oil (66 mg, 52% yield, FIG. 2).

¹H NMR (400 MHz, CDCl₃, 23 °C): δ 7.48-7.45 (m, 2H), 7.41-7.33 (m, 4H), 7.02-7 (m, 2H), 6.85-6.84 (m, 1H), 6.82-6.80 (m, 1H), 6.34-6.32 (m, 1H).

¹³C NMR (101 MHz, CDCl₃, 23 °C): δ 148.2, 148.0, 138.6, 134.5, 132.9, 132.8, 128.9, 128.7, 128.3, 128.2, 123.4, 121.5 (t, J=27.4 Hz), 119.7, 117.5 (t, J=35.4 Hz), 114.7 (m), 113.4 (m), 112.4 (t, J=37.8 Hz), 108.9.

¹⁹F NMR (376 MHz, CDCl₃, 23 °C): δ −99.3 (2F), −107.0 (2F).

HRMS (ESI) calculated for [M*H]⁺, C₁₈H₁₁F₄OBrClN: m/z 449.9701; found, 449.9357.

### Example 37

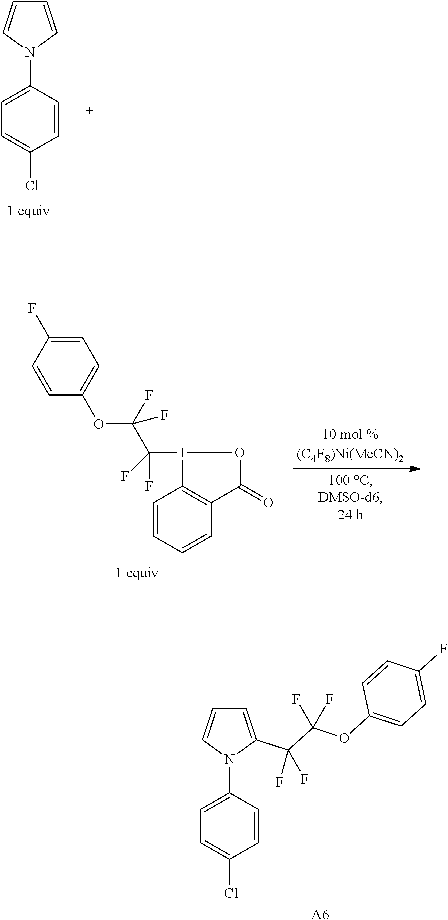 [SMILES: Clc1ccc(-n2cccc2)cc1 ; O=C1OI(C(F)(F)C(F)(F)Oc2ccc(F)cc2)c2ccccc21 ; Fc1ccc(OC(F)(F)C(F)(F)c2cccn2-c2ccc(Cl)cc2)cc1 ; "A6"]

A septum vial equipped with a magnetic stirring bar was charged with 1-(4-Chlorophenyl)-1H-pyrrole (10 mg, 0.056 mmol), 1-(2-(4-Fluorophenoxy)-1,1,2,2-tetrafluoroethyl)-1λ³-benzol[d][1,2]iodaoxol-3(1H)-one (25.7 mg, 0.056 mmol) and (C₄F₈)Ni(MeCN)₂ (1.9 mg, 0.0056 mmol), and then dissolved in anhydrous deuterated DMSO (1 mL). The reaction mixture was transferred to an oil bath at 100 °C for 24 hours. After reaction completion, α,α,α-trifluorotoluene (6.9 μL, 0.069 mmol) was added as a standard and the yield was determined by ¹⁹F NMR spectroscopy. The yield was 60% (FIG. 2).

### Example 38

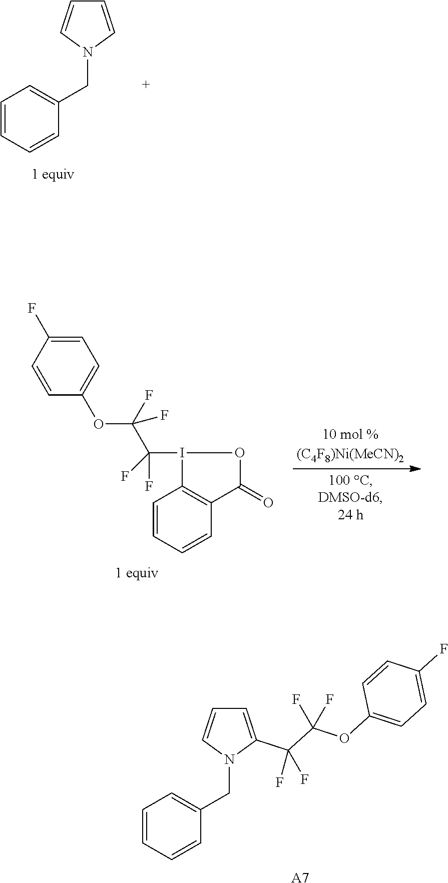 [SMILES: c1ccc(Cn2cccc2)cc1 ; O=C1OI(C(F)(F)C(F)(F)Oc2ccc(F)cc2)c2ccccc21 ; Fc1ccc(OC(F)(F)C(F)(F)c2cccn2Cc2ccccc2)cc1 ; "A7"]

A septum vial equipped with a magnetic stirring bar was charged with 1-Benzyl-pyrrole (7.7 μL, 0.05 mmol), 1-(2-(4-Fluorophenoxy)-1,1,2,2-tetrafluoroethyl)-1λ³-benzo[d][1,2]iodaoxol-3(1H)-one (22.9 mg, 0.05 mmol) and (C₄F₈)Ni(MeCN)₂ (1.7 mg, 0.005 mmol), and then dissolved in anhydrous deuterated DMSO (1 mL). The reaction mixture was transferred to an oil bath at 100 °C for 24 hours. After reaction completion, α,α,α-trifluorotoluene (6.1 L, 0.05 mmol) was added as a standard and the yield was determined by ¹⁹F NMR spectroscopy. The yield was 38% (FIG. 2).

### Example 39

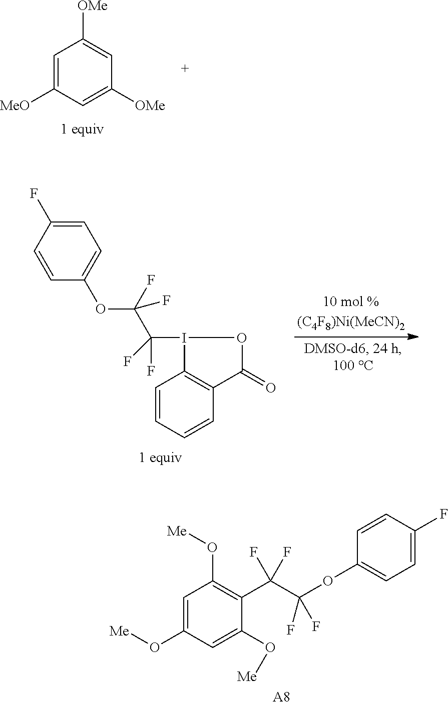

A septum vial equipped with a magnetic stirring bar was charged with 1,3,5-Trimethoxybenzene (10 mg, 0.059 mmol), 1-(2-(4-Fluorophenoxy)-1,1,2,2-tetrafluoroethyl)-1λ³-benzo[d][1,2]iodaoxol-3(1H)-one (27.2 mg, 0.059 mmol) and (C₄F₈)Ni(MeCN)₂ (2 mg, 0.0059 mmol), and then dissolved in anhydrous deuterated DMSO (1 mL). The reaction mixture was transferred to an oil bath at 100 °C for 24 hours. After reaction completion, α,α,α-trifluorotoluene (7.3 μL, 0.059 mmol) was added as a standard and the yield was determined by ¹⁹F NMR spectroscopy. The yield was 35% (FIG. 2).

### Example 40

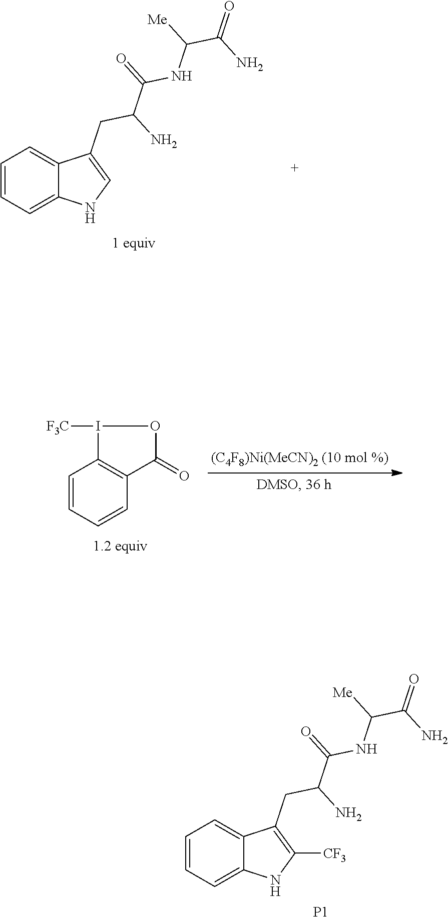 [SMILES: CC(NC(=O)C(N)Cc1c[nH]c2ccccc12)C(N)=O ; O=C1OI(C(F)(F)F)c2ccccc21 ; CC(NC(=O)C(N)Cc1c(C(F)(F)F)[nH]c2ccccc12)C(N)=O ; "P1"]

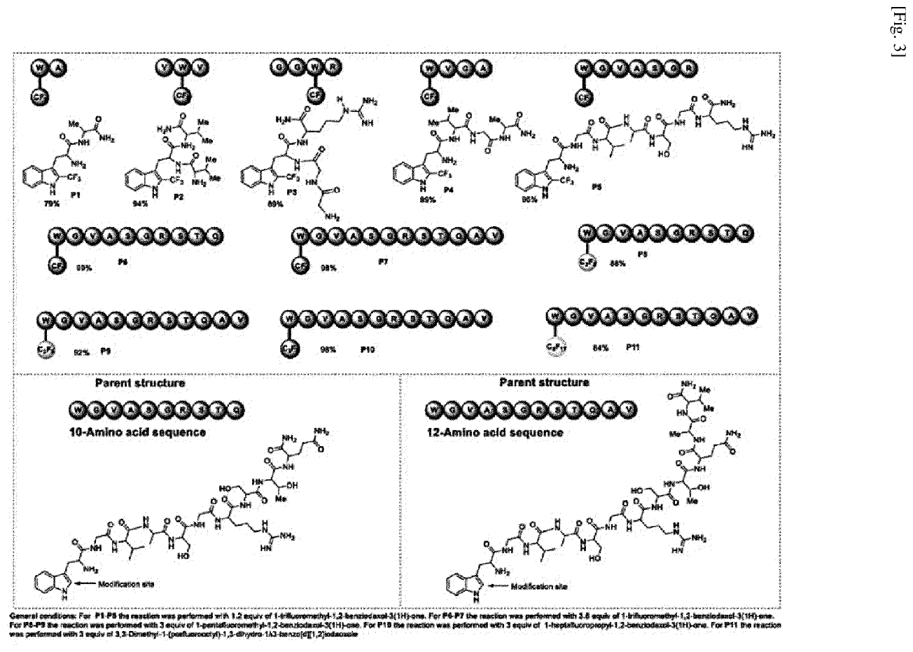

A 5 mL vial equipped with a magnetic stirring bar was charged with peptide (W-A-NH₂) (5 mg, 0.018 mmol), 1-trifluoromethyl-1,2-benziodoxol-3(1H)-one (17.2 mg, 0.021 mmol) and (C₄F₈)Ni(MeCN)₂ (0.6 mg, 0.001 mmol), which were dissolved in anhydrous DMSO (0.5 mL). Further the reaction mixture was stirred at room temperature for 36 hours. After reaction completion, the reaction mixture was analyzed by LC-MS. The yield was 79% (FIG. 3).

Note: the nickel precursor was added to the reaction mixture by preparing a stock solution in DMSO.

### Example 41

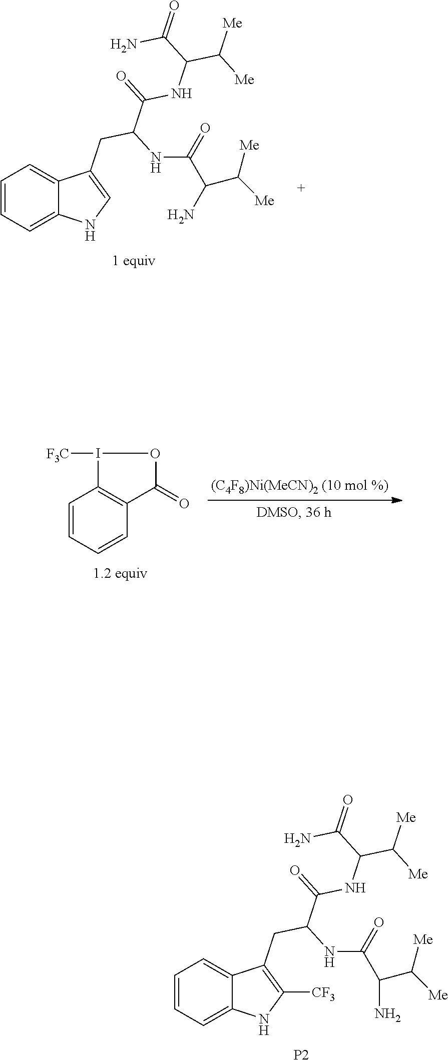

A 5 mL vial equipped with a magnetic stirring bar was charged with peptide (V-W-V-NH₂) (4.2 mg, 0.010 mmol), 1-trifluoromethyl-1,2-benziodoxol-3(1H)-one (9.9 mg, 0.012 mmol) and (C₄F₈)Ni(MeCN)₂ (0.3 mg, 0.001 mmol), which were dissolved in anhydrous DMSO (0.5 mL). Further the reaction mixture was stirred at room temperature for 36 hours. After reaction completion, the reaction mixture was analyzed by LC-MS. The yield was 94% (FIG. 3).

Note: the nickel precursor was added to the reaction mixture by preparing a stock solution in DMSO.

### Example 42

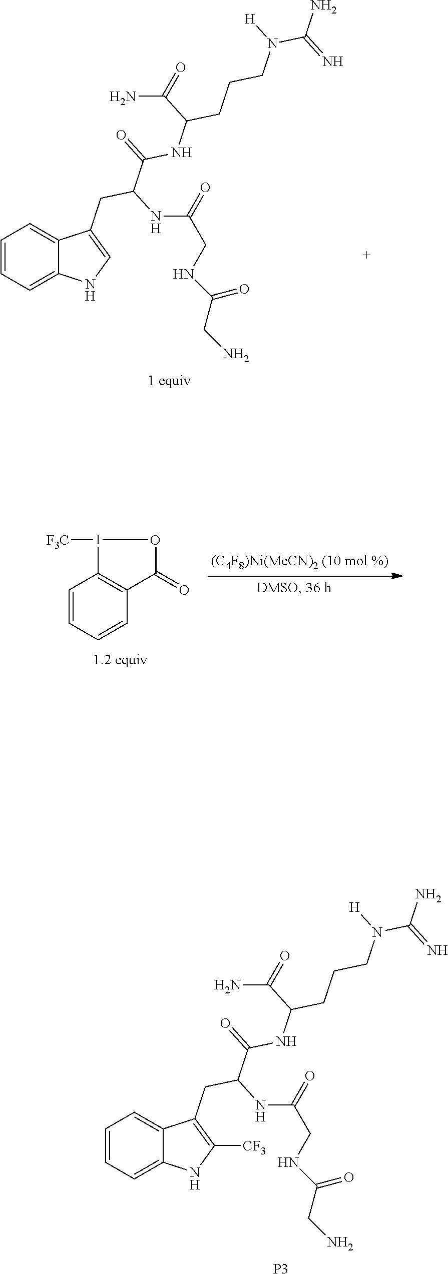 [SMILES: N=C(N)NCCCC(NC(=O)C(Cc1c[nH]c2ccccc12)NC(=O)CNC(=O)CN)C(N)=O ; O=C1OI(C(F)(F)F)c2ccccc21 ; N=C(N)NCCCC(NC(=O)C(Cc1c(C(F)(F)F)[nH]c2ccccc12)NC(=O)CNC(=O)CN)C(N)=O ; "P3"]

A 5 mL vial equipped with a magnetic stirring bar was charged with peptide (G-G-W-R—NH₂) (6.7 mg, 0.014 mmol), 1-trifluoromethyl-1,2-benziodoxol-3(1H)-one (13.4 mg, 0.016 mmol) and (C₄F₈)Ni(MeCN)₂ (0.4 mg, 0.001 mmol), which were dissolved in anhydrous DMSO (1 mL). Further the reaction mixture was stirred at room temperature for 36 hours. After reaction completion, the reaction mixture was analyzed by LC-MS. The yield was 89% (FIG. 3).

Note: the nickel precursor was added to the reaction mixture by preparing a stock solution in DMSO.

### Example 43

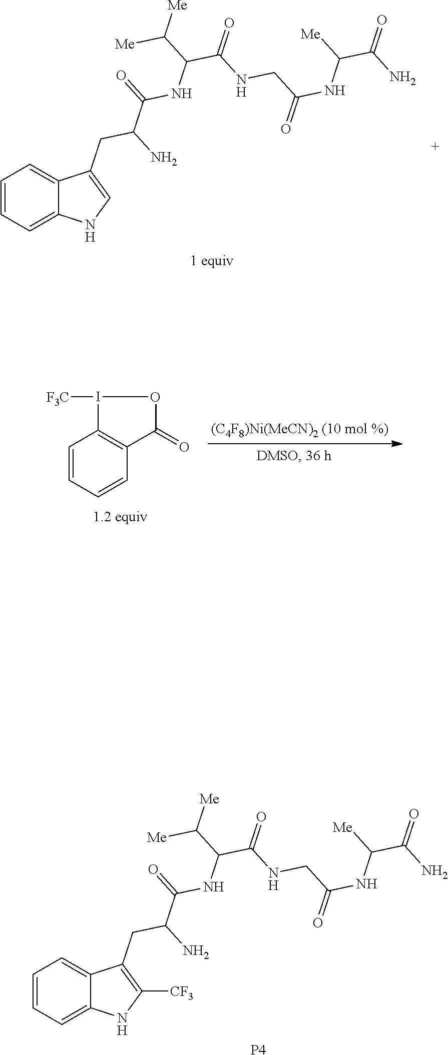 [SMILES: CC(NC(=O)CNC(=O)C(NC(=O)C(N)Cc1c[nH]c2ccccc12)C(C)C)C(N)=O ; "1 equiv" ; O=C1OI(C(F)(F)F)c2ccccc21 ; CC(NC(=O)CNC(=O)C(NC(=O)C(N)Cc1c(C(F)(F)F)[nH]c2ccccc12)C(C)C)C(N)=O ; "P4"]

A 5 mL vial equipped with a magnetic stirring bar was charged with peptide (W-V-G-A-NH₂) (5 mg, 0.011 mmol), 1-trifluoromethyl-1,2-benziodoxol-3(1H)-one (11 mg, 0.013 mmol) and (C₄F₈)Ni(MeCN)₂ (0.3 mg, 0.001 mmol), which were dissolved in anhydrous DMSO (1 mL). Further the reaction mixture was stirred at room temperature for 36 hours. After reaction completion, the reaction mixture was analyzed by LC-MS. The yield was 99% (FIG. 3).

Note: the nickel precursor was added to the reaction mixture by preparing a stock solution in DMSO.

### Example 44

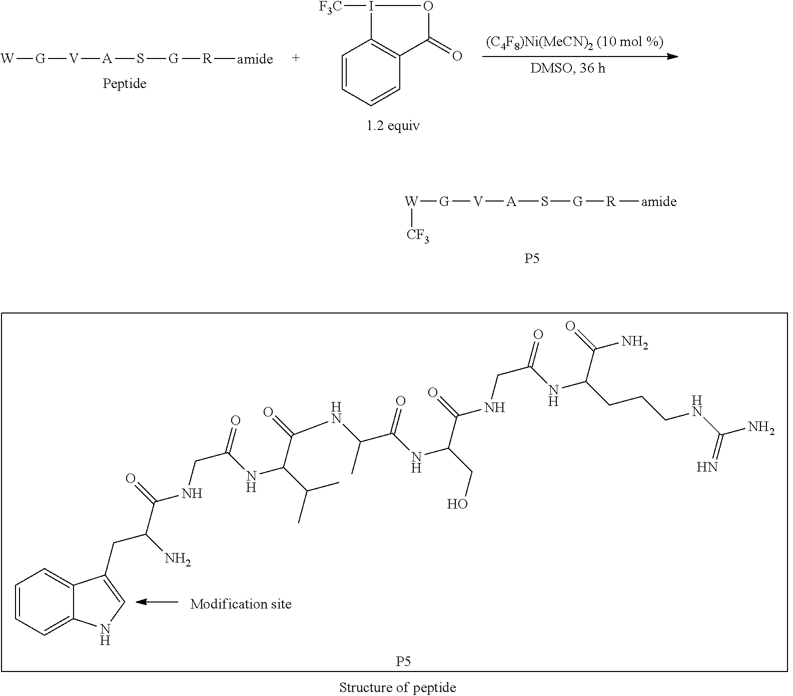

A 5 mL vial equipped with a magnetic stirring bar was charged with peptide (W-G-V-A-S-G-R—NH₂) (7 mg, 0.009 mmol), 1-trifluoromethyl-1,2-benziodoxol-3(1H)-one (9 mg, 0.011 mmol) and (C₄F₈)Ni(MeCN)₂ (0.3 mg, 0.0009 mmol), which were dissolved in anhydrous DMSO (1 mL). Further the reaction mixture was stirred at room temperature for 36 hours. After reaction completion, the reaction mixture was analyzed by LC-MS. The yield was 96% (FIG. 3).

Note: the nickel precursor was added to the reaction mixture by preparing a stock solution in DMSO.

### Example 45

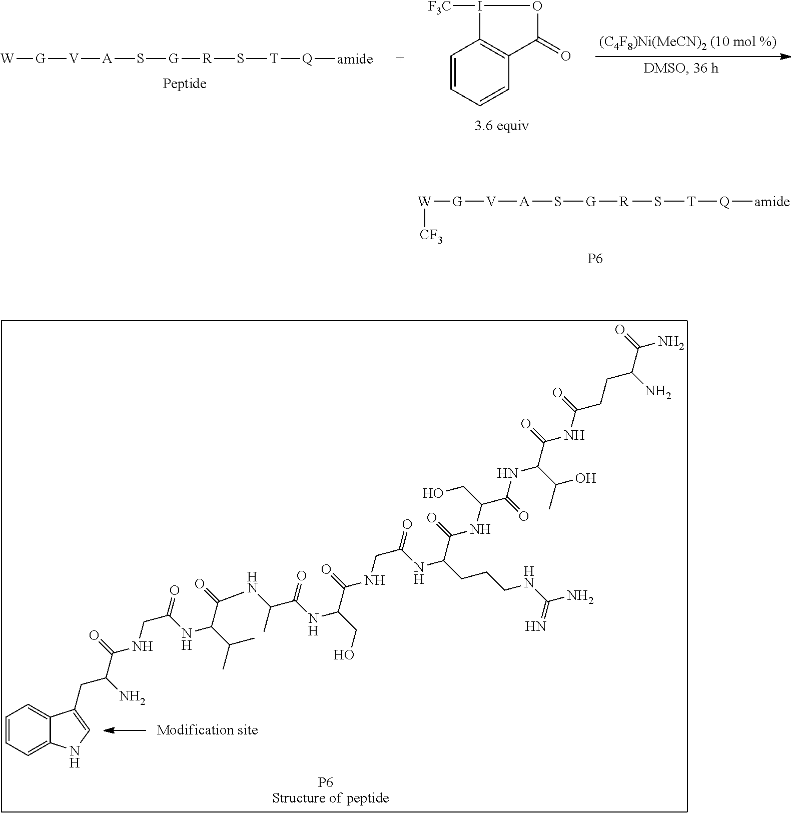

A 5 mL vial equipped with a magnetic stirring bar was charged with peptide (W-G-V-A-S-G-R-S-T-Q-NH₂) (5 mg, 0.004 mmol), 1-trifluoromethyl-1,2-benziodoxol-3(1H)-one (13.5 mg, 0.014 mmol) and (C₄F₈)Ni(MeCN)₂ (0.1 mg, 0.0004 mmol), which were dissolved in anhydrous DMSO (1 mL). Further the reaction mixture was stirred at room temperature for 36 hours. After reaction completion, the reaction mixture was analyzed by LC-MS. The yield was 99% (FIG. 3).

Note: the nickel precursor was added to the reaction mixture by preparing a stock solution in DMSO.

### Example 46

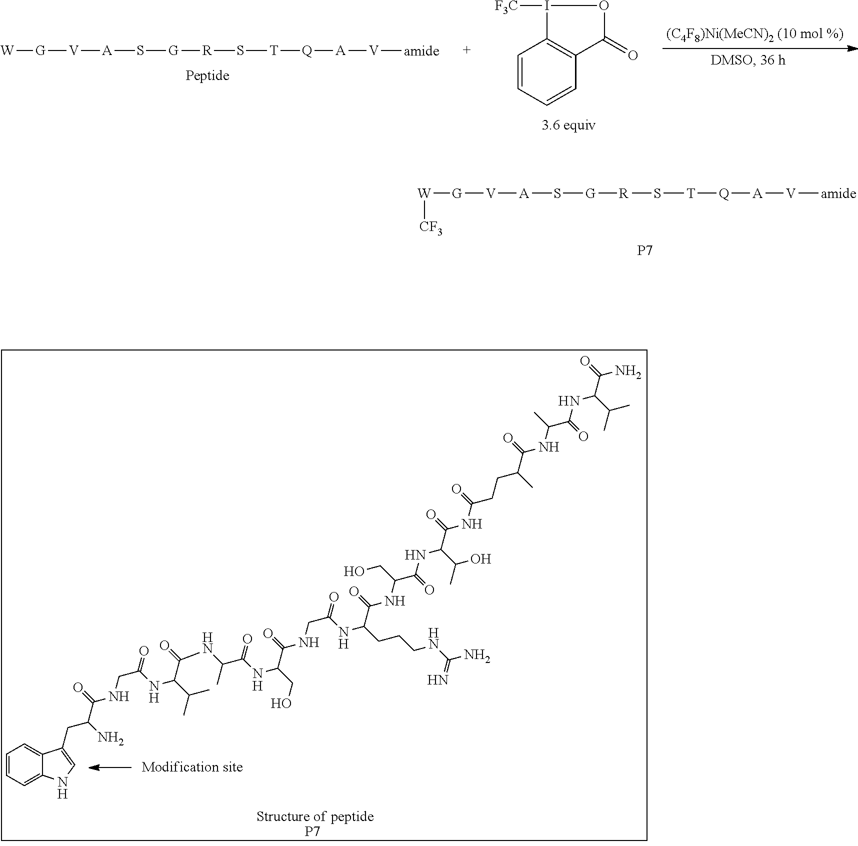

A 5 mL vial equipped with a magnetic stirring bar was charged with peptide (W-G-V-A-S-G-R-S-T-Q-A-V—NH₂) (5 mg, 0.004 mmol), 1-trifluoromethyl-1,2-benziodoxol-3(1H)-one (11.6 mg, 0.014 mmol) and (C₄F₈)Ni(MeCN)₂ (0.1 mg, 0.0004 mmol), which were dissolved in anhydrous DMSO (1 mL). Further the reaction mixture was stirred at room temperature for 36 hours. After reaction completion, the reaction mixture was analyzed by LC-MS. The yield was 98% (FIG. 3).

Note: the nickel precursor was added to the reaction mixture by preparing a stock solution in DMSO.

### Example 47

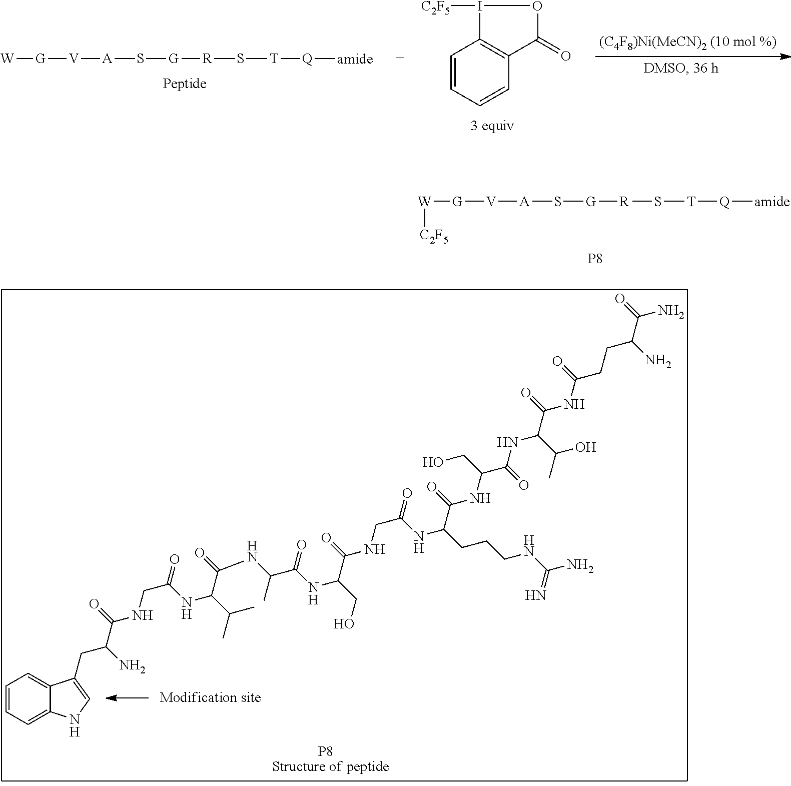

A 5 mL vial equipped with a magnetic stirring bar was charged with peptide (W-G-V-A-S-G-R-S-T-Q-NH₂) (5 mg, 0.004 mmol), 1-pentafluoroethyl-1,2-benziodoxol-3(1H)-one (5.1 mg, 0.014 mmol) and (C₄F₈)Ni(MeCN)₂ (0.1 mg, 0.0004 mmol), which were dissolved in anhydrous DMSO (1 mL). Further the reaction mixture was stirred at room temperature for 36 hours. After reaction completion, the reaction mixture was analyzed by LC-MS. The yield was 88% (FIG. 3).

Note: the nickel precursor was added to the reaction mixture by preparing a stock solution in DMSO.

### Example 48

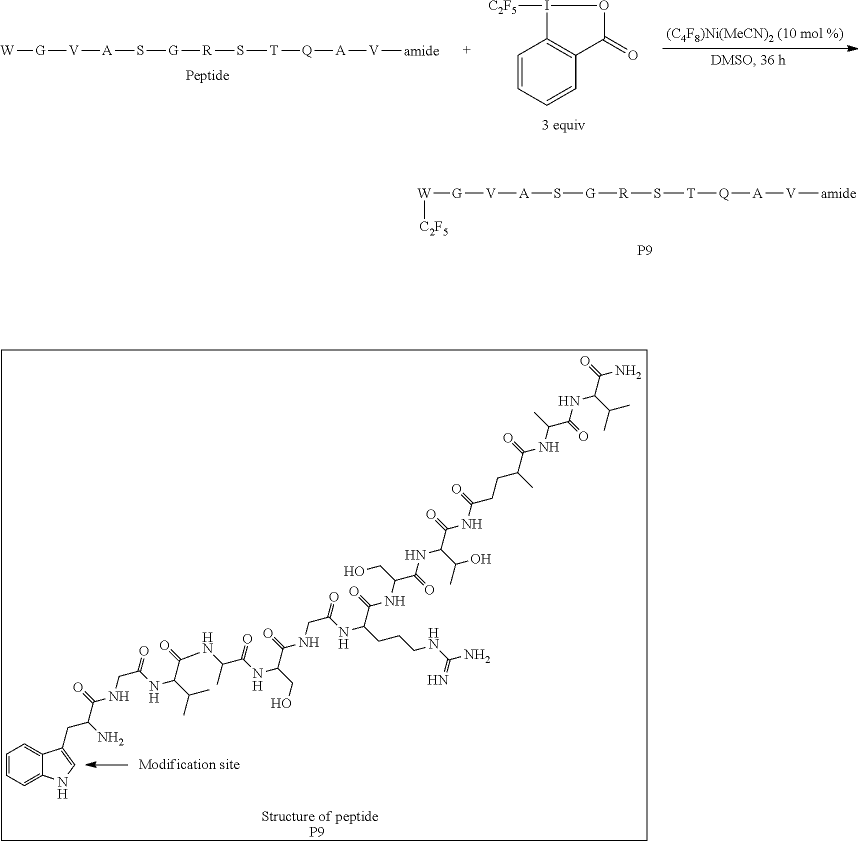

A 5 mL vial equipped with a magnetic stirring bar was charged with peptide (W-G-V-A-S-G-R-S-T-Q-A-V—NH₂) (5 mg, 0.004 mmol), 1-pentafluoro-1,2-benziodoxol-3(1H)-one (4.5 mg, 0.012 mmol) and (C₄F₈)Ni(MeCN)₂ (0.1 mg, 0.0004 mmol), which were dissolved in anhydrous DMSO (1 mL). Further the reaction mixture was stirred at room temperature for 36 hours. After reaction completion, the reaction mixture was analyzed by LC-MS. The yield was 92% (FIG. 3).

Note: the nickel precursor was added to the reaction mixture by preparing a stock solution in DMSO.

### Example 49

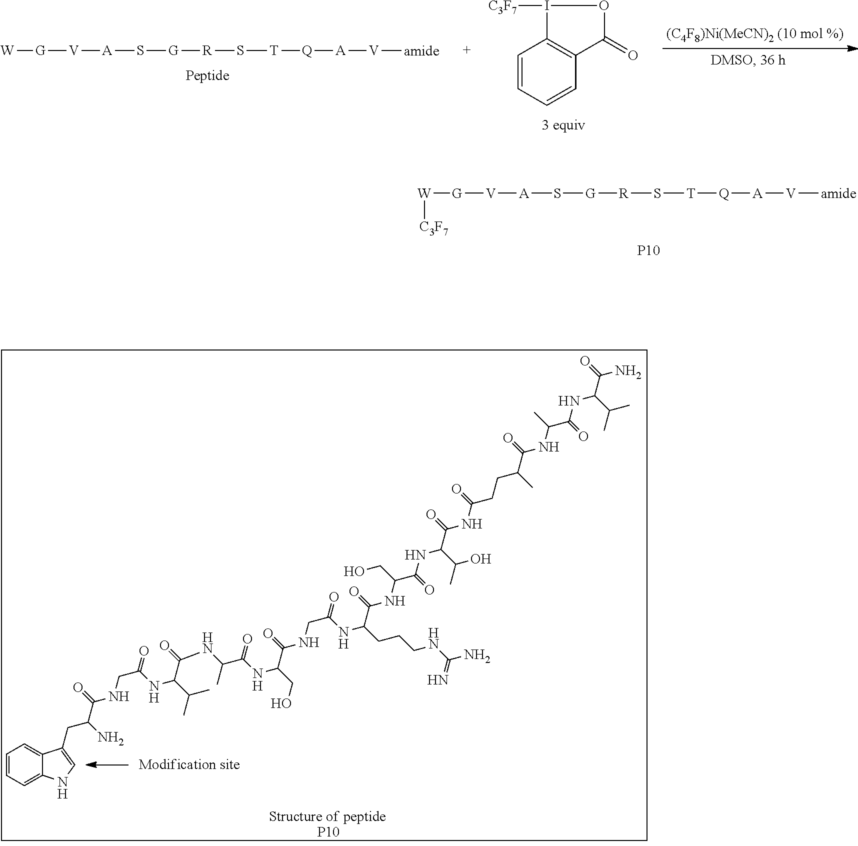

A 5 mL vial equipped with a magnetic stirring bar was charged with peptide (W-G-V-A-S-G-R-S-T-Q-A-V—NH₂) (5 mg, 0.004 mmol), 1-heptafluoropropyl-1,2-benziodoxol-3(1H)-one (5.1 mg, 0.012 mmol) and (C₄F₈)Ni(MeCN)₂ (0.1 mg, 0.0004 mmol), which were dissolved in anhydrous DMSO (1 mL). Further the reaction mixture was stirred at room temperature for 36 hours. After reaction completion, the reaction mixture was analyzed by LC-MS. The yield was 98% (FIG. 3).

Note: the nickel precursor was added to the reaction mixture by preparing a stock solution in DMSO.

### Example 50

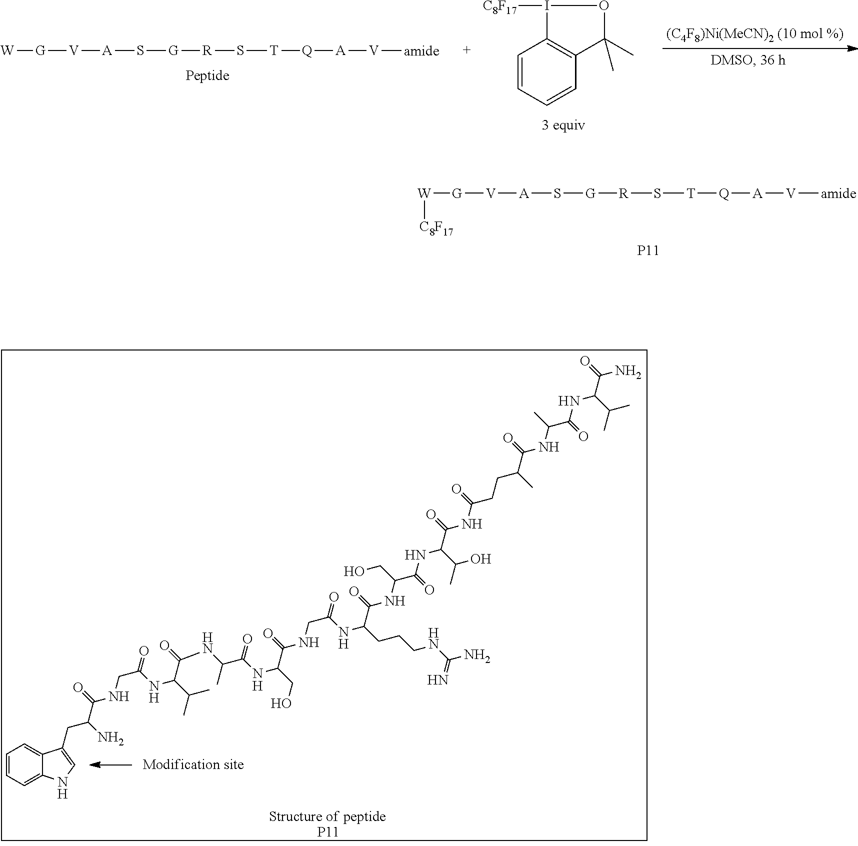

A 5 mL vial equipped with a magnetic stirring bar was charged with peptide (W-G-V-A-S-G-R-S-T-Q-A-V—NH₂) (5 mg, 0.004 mmol), 3,3-Dimethyl-1-(perfluorooctyl)-1,3-dihydro-1λ³-benzo[d][1,2]iodaoxole (8.3 mg, 0.012 mmol) and (C₄F₈)Ni(MeCN)₂ (0.1 mg, 0.0004 mmol), which were dissolved in anhydrous DMSO (1 mL). Further the reaction mixture was stirred at room temperature for 36 hours. After reaction completion, the reaction mixture was analyzed by LC-MS. The yield was 84% (FIG. 3).

Note: the nickel precursor was added to the reaction mixture by preparing a stock solution in DMSO.

### INDUSTRIAL APPLICABILITY

According to the present disclosure, it is possible to provide a catalyst that can be used in a variety of reactions involving a formation of a carbon-carbon bond.

Moreover, according to the present disclosure, it is possible to provide a modified compound that can be prepared by a reaction involving a formation of a carbon-carbon bond.

Furthermore, according to the present disclosure, it is possible to provide a method of preparing a modified compound that can be prepared by a reaction involving a formation of a carbon-carbon bond.

1. A catalyst used in a reaction involving a formation of a carbon-carbon bond, wherein the catalyst is represented by a formula (I): 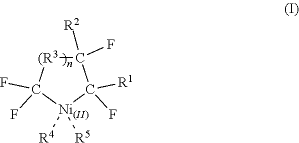
wherein
R¹ is selected from the group consisting of a hydrogen atom, a fluorine atom, a hydrocarbon group and a heterocyclic group;
R² is selected from the group consisting of a hydrogen atom, a fluorine atom, a hydrocarbon group and a heterocyclic group;
each R³ is independently selected from the group consisting of CH₂, CHF and CF₂;
R⁴ and R⁵ are the same or different and each is a polar organic solvent comprising at least one selected from a nitrogen atom, an oxygen atom, a sulfur atom and a halogen atom;
n is an integer larger than 0; and
dashed lines connecting Ni (II) and R⁴ and R⁵ are coordinate bonds.

2. The catalyst according to claim 1, wherein R¹ is a fluorine atom.

3. The catalyst according to claim 1, wherein R² is a fluorine atom.

4. The catalyst according to claim 1, wherein each R³ is CF₂.

5. The catalyst according to claim 1, wherein R⁴ and R⁵ are independently selected from the group consisting of an alcohol, an amide, a ketone, an ether, a nitrile, an alkyl halide and a sulfoxide.

6. The catalyst according to claim 1, wherein both R⁴ and R⁵ are CH₃CN.

7. The catalyst according to claim 1, wherein n is an integer from 1 to 3.

8. The catalyst according to claim 1, wherein the carbon-carbon bond is formed between a carbon atom of a substrate and a fluorocarbon group of a fluorocarbon compound.

9. The catalyst according to claim 8, wherein the substrate comprises at least one of an aromatic carbon ring and an aromatic heterocyclic ring.

10. The catalyst according to claim 8, wherein the fluorocarbon compound comprises at least one —(CF₂)ₘ—, wherein m is an integer larger than 0.

11. The catalyst according to claim 8, wherein the fluorocarbon compound is represented by formula (II-1), (II-2), (II-3) or (II-4): 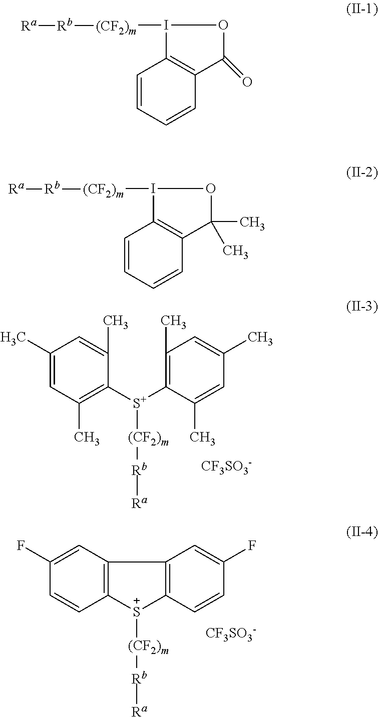
wherein
R^{a} is selected from the group consisting of a fluorine atom, an aryl group and a heteroaryl group, wherein the aryl group and the heteroaryl group are each optionally substituted by one or more substituents selected from the group consisting of a heteroatom and an alkyl group;
R^{b} is a single bond or an oxygen atom; and
m is an integer larger than 0.

12. A modified compound prepared by a reaction involving a formation of a carbon-carbon bond, wherein the reaction uses a catalyst represented by a formula (I): 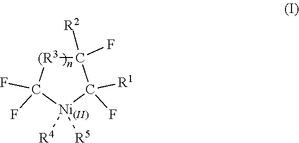
wherein
R¹ is selected from the group consisting of a hydrogen atom, a fluorine atom, a hydrocarbon group and a heterocyclic group;
R² is selected from the group consisting of a hydrogen atom, a fluorine atom, a hydrocarbon group and a heterocyclic group;
each R³ is independently selected from the group consisting of CH₂, CHF and CF₂;
R⁴ is a polar organic solvent comprising at least one selected from a nitrogen atom, an oxygen atom, a sulfur atom and a halogen atom;
R⁵ is a polar organic solvent comprising at least one selected from a nitrogen atom, an oxygen atom, a sulfur atom and a halogen atom;
n is an integer larger than 0; and
dashed lines connecting Ni (II) and R⁴ and R⁵ are coordinate bonds.

13. A method of preparing a modified compound represented by formula (IV), comprising:
reacting a substrate represented by formula (III) with a fluorocarbon compound represented by formula (II-1), (II-2), (II-3) or (II-4), in the presence of the catalyst according to claim 1 to obtain the modified compound represented by formula (IV): 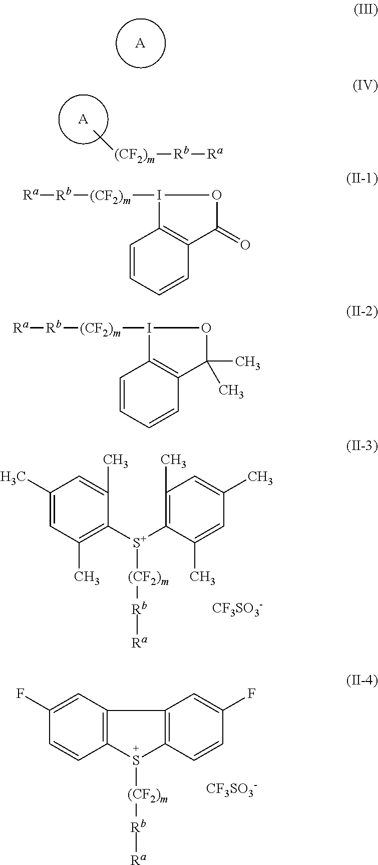
wherein
A is selected from the group consisting of an aromatic carbon ring and an aromatic heterocyclic ring, wherein the aromatic carbon ring and the aromatic heterocyclic ring may be substituted by one or more substituents;
A may be a part of a peptide or protein;
R^{a} is selected from the group consisting of a fluorine atom, an aryl group and a heteroaryl group, wherein the aryl group and the heteroaryl group are each optionally substituted by one or more substituents selected from the group consisting of a heteroatom and an alkyl group:
R^{b} is a single bond or an oxygen atom; and
m is an integer larger than 0.

14. The method according to claim 13, wherein A is selected from the group consisting of a benzene ring and an aromatic heterocyclic ring having 5 to 12 ring atoms.

15. The method according to claim 13, wherein A is an indole ring of a tryptophan residue in a peptide or a protein.
